(19) United States
(12) Patent Application Publication
(10) Pub. No.: US 2020/0320697 A1
(43) Pub. Date: Oct. 8, 2020

(54) METHOD, SYSTEM, AND DEVICE FOR LUNG LOBE SEGMENTATION, MODEL TRAINING, MODEL CONSTRUCTION AND SEGMENTATION

(71) Applicant: Alibaba Group Holding Limited, Grand Cayman (KY)

(72) Inventors: Wenjia Wang, Beijing (CN); Junxuan Chen, Beijing (CN); Ying Chi, Beijing (CN); Xuansong Xie, Beijing (CN); Xiansheng Hua, Hangzhou (CN)

(21) Appl. No.: 16/839,886

(22) Filed: Apr. 3, 2020

(30) Foreign Application Priority Data
Apr. 4, 2019 (CN) 201910273160.6

(51) Int. Cl. G06T 7/00; G06T 5/00; G06T 7/11; G06T 5/20; G06T 7/136; G06N 3/08; G16H 30/40

(52) U.S. Cl. CPC G06T 7/0012; G06T 5/008; G06T 7/11; G06T 5/20; G06T 7/136; G06T 2207/20084; G16H 30/40; G06T 2207/30061; G06T 2207/20081; G06T 2207/20152; G06N 3/08

(57) ABSTRACT

A method includes: determining a first neural network model; inputting an image to be processed containing a lung image to the first neural network model to obtain a lung lobe segmentation result of the image to be processed; wherein the first network layer in the first neural network model is configured to add an additional channel with coordinate information to input data input to the first network layer, and to determine output data of the first network layer based on the input data after the additional channel is added thereto.

### CROSS REFERENCE TO RELATED PATENT APPLICATIONS

This application claims priority to Chinese Patent Application No. 201910273160.6, filed on Apr. 4, 2019, entitled “METHOD, SYSTEM, AND DEVICE FOR LUNG LOBE SEGMENTATION, MODEL TRAINING, MODEL CONSTRUCTION AND SEGMENTATION,” which is hereby incorporated by reference in its entirety.

### TECHNICAL FIELD

The present disclosure relates to the field of computer technology, and in particular, to methods, systems, and devices for lung lobe segmentation, model training, model construction and segmentation.

### BACKGROUND

Image recognition using neural network models is a commonly used technical measure in the field of artificial intelligence. Image recognition refers to identifying a target object in an image or achieving segmentation of a target object in an image, such as animal body segmentation or animal body organ segmentation, and the like.

Taking human lung lobe segmentation as an example, in a medical institution such as a hospital, a physical examination center, and the like, after detecting a patient's lung disease (for example, a pulmonary nodule, pleural effusion), the lung lobe segmentation information needs to be used for positioning, thereby facilitating surgical planning. With the development of computer vision recognition technology, many hospitals and physical examination centers use deep learning models to obtain lung lobe segmentation information, that is, the lung is divided into five lung lobes (left upper lung lobe, left lower lung lobe, right upper lung lobe, right middle lung lobe, and right lower lung lobe).

However, with current deep learning models, misclassification often occurs when the target object, such as the lung lobe, is segmented.

### SUMMARY

This Summary is provided to introduce a selection of implementations in a simplified form that are further described below in the Detailed Description. This Summary is not intended to identify all features of the claimed subject matter, nor is it intended to be used alone as an aid in determining the scope of the claimed subject matter. The term “techniques,” for instance, may refer to device(s), system(s), method(s) and/or processor-readable/computer-readable instructions as permitted by the context above and throughout the present disclosure.

In view of the above problems, the present disclosure is proposed to provide methods, systems, and devices for lung lobe segmentation, model training, model construction and segmentation that address the above problems or at least partially address the above problems.

Therefore, in an example embodiment of the present disclosure, a lung lobe segmentation method is provided. The method includes:

determining a first neural network model; and

inputting an image to be processed containing a lung image to the first neural network model to obtain a lung lobe segmentation result of the image to be processed;

wherein the first network layer in the first neural network model is configured to add an additional channel with coordinate information to input data input to the first network layer, and to determine output data of the first network layer based on the input data after the additional channel is added thereto.

In another example embodiment of the present disclosure, a model training method is provided. The method includes:

inputting a sample image containing a lung image to a first neural network model to obtain a lung lobe segmentation prediction result of the sample image; and

performing parameter optimization on the first neural network model according to the lung lobe segmentation prediction result and a lung lobe segmentation ground truth result of the sample image;

wherein the first neural network model is configured for lung lobe segmentation; a first network layer in the first neural network model is configured to add an additional channel with coordinate information to input data input to the first network layer, and to determine output data of the first network layer based on the input data to which the additional channel is added.

In yet another example embodiment of the present disclosure, a model construction method is provided. The method includes:

constructing at least one network layer to obtain a first neural network model for lung lobe segmentation;

wherein the at least one network layer includes at least one first network layer; and the first network layer is configured to add an additional channel with coordinate information to input data input to the first network layer, and to determine output data of the first network layer based on the input data after the additional channel is added thereto.

In yet another example embodiment of the present disclosure, a lung lobe segmentation method is provided. The method includes:

inputting an image to be processed containing a lung image to a second neural network model to obtain a lung segmentation result, wherein the lung segmentation result includes an identified lung region;

inputting the image to be processed to a first neural network model to obtain a lung lobe segmentation result of the image to be processed; and

modifying the lung lobe segmentation result according to the lung segmentation result.

In yet another example embodiment of the present disclosure, a segmentation method is provided. The method includes:

inputting an image to be processed containing a target object image to a second neural network model to obtain a target segmentation result, wherein the target segmentation result includes an identified target object region;

inputting the image to be processed to a first neural network model to obtain an identified unit region corresponding to a unit constituting the target object; and

modifying the unit region according to the target segmentation result.

In yet another example embodiment of the present disclosure, a neural network system is provided. The system includes multiple network layers for identifying a unit region corresponding to a unit constituting a target object in an image to be processed; wherein in any two of the multiple network layers that are connected to each other, input data of a downstream network layer is output data of an upstream network layer; and

wherein the multiple network layers include a first network layer; and the first network layer is configured to add an additional channel with coordinate information to input data input to the first network layer, and to determine output data of the first network layer based on the input data to which the additional channel is added.

In yet another example embodiment of the present disclosure, a segmentation method is provided. The method includes:

acquiring an image to be processed containing a target object image;

processing the image to be processed, wherein the processing procedure includes an operation of adding an additional channel with coordinate information; and

identifying a unit region corresponding to a unit constituting the target object based on a processing result of the image to be processed.

In yet another example embodiment of the present disclosure, an electronic device is provided. The device includes a memory and a processor, wherein,

the memory is configured to store a program;

the processor is coupled to the memory and is configured to execute programs stored in the memory, so as to:

determine a first neural network model; and

input an image to be processed containing a lung image to the first neural network model to obtain a lung lobe segmentation result of the image to be processed;

wherein a first network layer in the first neural network model is configured to add an additional channel with coordinate information to input data input to the first network layer, and to determine output data of the first network layer based on the input data after the additional channel is added thereto.

In yet another example embodiment of the present disclosure, an electronic device is provided. The device includes a memory and a processor, wherein,

the memory is configured to store a program; and

the processor is coupled to the memory, and is configured to execute the program stored in the memory, so as to:

input a sample image containing a lung image to a first neural network model to obtain a lung lobe segmentation prediction result of the sample image; and

perform parameter optimization on the first neural network model according to the lung lobe segmentation prediction result and a lung lobe segmentation ground truth result of the sample image;

wherein the first neural network model is configured for lung lobe segmentation; the first network layer in the first neural network model is configured to add an additional channel with coordinate information to input data input to the first network layer, and to determine output data of the first network layer based on the input data to which the additional channel is added.

In yet another example embodiment of the present disclosure, an electronic device is provided. The device includes a memory and a processor, wherein,

the memory is configured to store a program; and

the processor is coupled to the memory, and is configured to execute the program stored in the memory, so as to:

construct at least one network layer to obtain a first neural network model for lung lobe segmentation;

wherein the at least one network layer includes at least one first network layer; and the first network layer is configured to add an additional channel with coordinate information to input data input to the first network layer, and to determine output data of the first network layer based on the input data after the additional channel is added thereto.

In another example embodiment of the present disclosure, an electronic device is provided. The device includes a memory and a processor, wherein,

the memory is configured to store a program; and

the processor is coupled to the memory, and is configured to execute the program stored in the memory, so as to:

input an image to be processed containing a lung image to a second neural network model to obtain a lung segmentation result, wherein the lung segmentation result includes an identified lung region;

input the image to be processed to a first neural network model to obtain a lung lobe segmentation result of the image to be processed; and

modify the lung lobe segmentation result according to the lung segmentation result.

In yet another example embodiment of the present disclosure, an electronic device is provided. The device includes a memory and a processor, wherein,

the memory is configured to store a program; and

the processor is coupled to the memory, and is configured to execute the program stored in the memory, so as to:

input an image to be processed containing a target object image to a second neural network model to obtain a target segmentation result, wherein the target segmentation result includes an identified target object region;

input the image to be processed to a first neural network model to obtain an identified unit region corresponding to a unit constituting the target object; and

modifying the unit region according to the target segmentation result.

In another example embodiment of the present disclosure, an electronic device is provided. The device includes a memory and a processor, wherein,

the memory is configured to store a program; and

the processor is coupled to the memory, and is configured to execute the program stored in the memory, so as to:

acquire an image to be processed containing a target object image;

process the image to be processed, wherein the processing procedure includes an operation of adding an additional channel with coordinate information; and

identifying a unit region corresponding to a unit constituting the target object based on a processing result of the image to be processed.

In the technical solutions provided by the example embodiments of the present disclosure, the first network layer is designed in the first neural network model for lung lobe segmentation. The first network layer adds an additional channel with coordinate information to input data input to the first network layer, and to determine the output data of the first network layer based on the input data after the additional channel is added thereto. The relative position distribution of the five lung lobes in the lung image is relatively fixed. The introduction of coordinate information may enable the neural network model to learn the coordinate features and global information, which play the role of coordinate guidance, effectively reducing the misclassification within the lung, and improving the accuracy of lung lobe segmentation.

### BRIEF DESCRIPTION OF THE DRAWINGS

In order to describe the technical solutions in the example embodiments of the present disclosure more clearly, the drawings used in descriptions of the example embodiments or the conventional techniques will be briefly introduced hereinafter. Drawings in the following descriptions merely represent some example embodiments of the present disclosure. For those of ordinary skill in the art, other drawings can be obtained based on these drawings without making creative efforts.

FIG. 1A, FIG. 1B, FIG. 1C, and FIG. 1D are schematic flowcharts of a lung lobe segmentation method provided by an example embodiment of the present disclosure;

FIG. 2A and FIG. 2B are schematic flowcharts of a model training method provided by an example embodiment of the present disclosure;

FIG. 3A and FIG. 3B are schematic flowcharts of a lung lobe segmentation method provided by another example embodiment of the present disclosure;

FIG. 4A and FIG. 4B are schematic flowcharts of a segmentation method provided by an example embodiment of the present disclosure;

FIG. 5 is a structural block diagram of a lung lobe segmentation apparatus provided by an example embodiment of the present disclosure;

FIG. 6 is a structural block diagram of a model training apparatus provided by an example embodiment of the present disclosure;

FIG. 7 is a structural block diagram of a segmentation apparatus provided by an example embodiment of the present disclosure;

FIG. 8 is a structural block diagram of a lung lobe segmentation apparatus provided by another example embodiment of the present disclosure;

FIG. 9 is a structural block diagram of a segmentation apparatus provided by another example embodiment of the present disclosure;

FIG. 10 is a structural block diagram of an electronic device provided by another example embodiment of the present disclosure; and

FIG. 11A, FIG. 11B, FIG. 11C, and FIG. 11D are schematic flowcharts of a segmentation method provided by another example embodiment of the present disclosure.

FIG. 12A and FIG. 12B are schematic flowcharts of a generating process of additional channels provided by an example embodiment of the present disclosure.

FIG. 13 is a schematic flowchart of a method for constructing the first neural network model provided by an example embodiment of the present disclosure.

FIG. 14 illustrates a structural block diagram of a model construction apparatus provided by an example embodiment of the present disclosure.

### DETAILED DESCRIPTION

To enable those skilled in the art to better understand the present disclosure, hereinafter, technical solutions in the example embodiments of the present disclosure will be clearly and thoroughly described with reference to the accompanying drawings in the example embodiments of the present disclosure. Example embodiments described herein merely illustrate some of the example embodiments of the present disclosure. Other example embodiments obtained by those skilled in the art based on the example embodiments of the present disclosure without making creative efforts should fall within the scope of the present disclosure.

Currently, multi-layered neural network models are often used for lung lobe segmentation, such as Convolutional Neural Networks (CNN) models. What needs to be noted is that the word segmentation in the example embodiments of the present disclosure can be understood as semantic segmentation.

A neural network model usually includes multiple layers of network layers. Each of the network layers includes multiple neurons (nodes). The multilayer network layer of the neural network model includes an input layer, an output layer, and at least one hidden layer between the input layer and the output layer (the respective layers other than the input layer and the output layer are referred to as hidden layers, and the hidden layer does not directly receive signals from the outside, or directly send signals to the outside). The at least one hidden layer can calculate and process the image to be processed input to the input layer step by step, to obtain an in-depth expression of the image to be processed. Therefore, the output of the hidden layer is a feature map that can represent the image to be processed. The feature maps output by different hidden layers are different. Each hidden layer represents the features of the previous hidden layer more deeply. The feature map of each of the hidden layers is usually a multi-channel feature map. The multi-channel feature map includes multiple channel maps. For example, when the image to be processed is a two-dimensional image, the corresponding multi-channel feature map can be expressed as a c*w*h structure, where c refers to the number of channels, and w and h refer to the width and the height of each channel map (at this time, the channel map is two-dimensional). When the image to be processed is a three-dimensional image, the corresponding multi-channel feature map can be expressed as c*d*w*h, where c refers to the number of channels, and d, w, and h refer to the depth, the width, and the height of each channel map (at this time the channel map is three-dimensional).

Currently, for the problem of lung lobe segmentation, some previous unsupervised methods include watershed transformation, graphic cutting, surface fitting, and semi-automatic segmentation. These methods use anatomical information as priori knowledge, including the distribution of airways, blood vessels, and fissures in the lungs, followed by the final segmentation of the lung lobes. However, the segmentation based on trachea and blood vessels is not always reliable, and these methods are less robust. Although with the development of computer vision, end-to-end deep learning models have emerged to segment five lung lobes, the existing deep learning models still have inaccurate segmentation problems when performing lung lobe segmentation.

While implementing the technical solution of the present disclosure, the inventor found through research that the relative position distribution of the five lung lobes in the lung image is relatively fixed. If coordinate information is introduced into the neural network model, the misclassification within the lung may be reduced. Accordingly, the inventor has proposed the technical solution of the present disclosure. In the technical solution provided in the example embodiments of the present disclosure, the first network layer is designed in the first neural network model for lung lobe segmentation. The first network layer in the first neural network model is used for adding an additional channel with coordinate information to the input data input to the first network layer, and determining output data of the first network layer based on the input data after the additional channel is added thereto. The relative position distribution of the five lung lobes in the lung image is relatively fixed. The introduction of coordinate information may enable the neural network model to learn the coordinate features and global information, which play the role of coordinate guidance, effectively reducing the misclassification within the lung, and improving the accuracy of segmentation.

What needs to be added is that the introduction of coordinate information can optimize the engineering performance, such as reducing the number of network parameters and reducing the prediction time.

In order to enable those of ordinary skill in the art to better understand the solutions of the present disclosure, the technical solutions in the example embodiments of the present disclosure will be clearly and completely described hereinafter with reference to the drawings in the example embodiments of the present disclosure. The described example embodiments are only some example embodiments of the present disclosure, but not all the example embodiments. Based on the example embodiments in the present disclosure, all other example embodiments obtained by those of ordinary skill in the art without making creative efforts fall into the protection scope of the present disclosure.

In addition, some processes described in the specification, claims, and above drawings of the present disclosure include a plurality of operations that occur in a particular order, and these operations may be performed in an order that is not the order appearing in the context. Otherwise, these operations may be performed in parallel. The sequence numbers of operations such as 101 and 102 are only used to distinguish different operations. The sequence numbers do not represent any order of execution. In addition, these processes may include more or fewer operations, and these operations may be performed sequentially or in parallel. What needs to be noted is that the descriptions such as “first”, “second”, and the like in the context are used to distinguish different messages, devices, modules, etc., and do not represent the sequence, nor do they limit “first” and “second” as different types.

An example embodiment of the present disclosure provides a neural network system. The neural network system includes: multiple network layers configured to identify a unit region corresponding to a unit constituting a target object in an image to be processed; where input data of a downstream network layer in any two network layers that are connected in the multiple network layers is output data of an upstream network layer; where the multiple network layers include the first network layer; the first network layer configured to add an additional channel with coordinate information to the input data input to the first network layer, and to determine the output data of the first network layer based on the input data to which the additional channel is added.

In actual applications, the image to be processed may be an image containing a biological body or a biological organ image acquired by a medical device (such as a CT device or an X-ray device), or an image containing an internal structure image of a device acquired by an industrial device (such as an industrial CT device), and the like. The example embodiments of the present disclosure are not limited herein. The target object may be an image of the object of interest in the image to be processed, for example, a device image with multiple constituent units (or structures), an animal body with multiple constituent units, an animal body organ, and the like.

There are usually multiple units constituting the target object. Therefore, the number of identified unit regions may be multiple, and each unit corresponds to a unit region.

The input data input to the first network layer may be an image to be processed or output data of an upstream network layer connected to the first network layer. For example, the output data of the upstream network layer connected to the first network layer may be the first feature map obtained after feature extraction is performed on the image to be processed.

In the technical solution provided by the example embodiments of the present disclosure, the first network layer is designed in a neural network system. The first network layer adds an additional channel with coordinate information to the input data input to the first network layer, and determines the output data of the first network layer based on the input data to which the additional channel is added. When the unit regions corresponding to the units constituting the target object in the image to be processed are identified, because the relative position distribution of the respective units constituting the target object is relatively fixed, the introduction of coordinate information may enable the neural network system to learn the coordinate features and global information, which play the role of coordinate guidance, effectively reducing the misclassification, and improving the accuracy of segmentation.

In an example, the input data input to the first network layer may include at least one channel map. When the input data includes two or more channel maps, the input data may be referred to as a multi-channel feature map. When only one channel map is included in the input data, the input data can be referred to as a single-channel feature map.

The first network layer adds additional channels with coordinate information to the input data. The number of additional channels is determined by the dimension of the image to be processed or the dimension of the channel map in the input data. The size of each additional channel is the same as the size of the channel map. When the image to be processed is a two-dimensional image, the channel map in the input data is also two-dimensional, and the number of additional channels is two, that is, the additional channel corresponding to the first axis (such as the x-axis) and the additional channel corresponding to the second axis (such as the y-axis), where the first axis and the second axis intersect. The two-dimensional size of each additional channel is the same as the two-dimensional size of the channel map. When the image to be processed is a three-dimensional image, the channel map in the input data is also three-dimensional, and the number of additional channels is three, that is, the additional channel corresponding to the first axis (such as the x axis), the additional channel corresponding to the second axis (such as the y axis), and the additional channel corresponding to the third axis (such as the z axis). The first axis, the second axis, and the third axis intersect with one another. The three-dimensional size of each additional channel is the same as the three-dimensional size of the channel map. The coordinates of the respective element on corresponding axes in the channel map in the input data are added to corresponding positions in the additional channels corresponding to respective axes.

In an implementable solution, the input data is a multi-channel feature map, that is, the input data includes at least two channel maps. The multi-channel feature map includes a first channel map. The first channel map refers to any channel map in at least two channel maps.

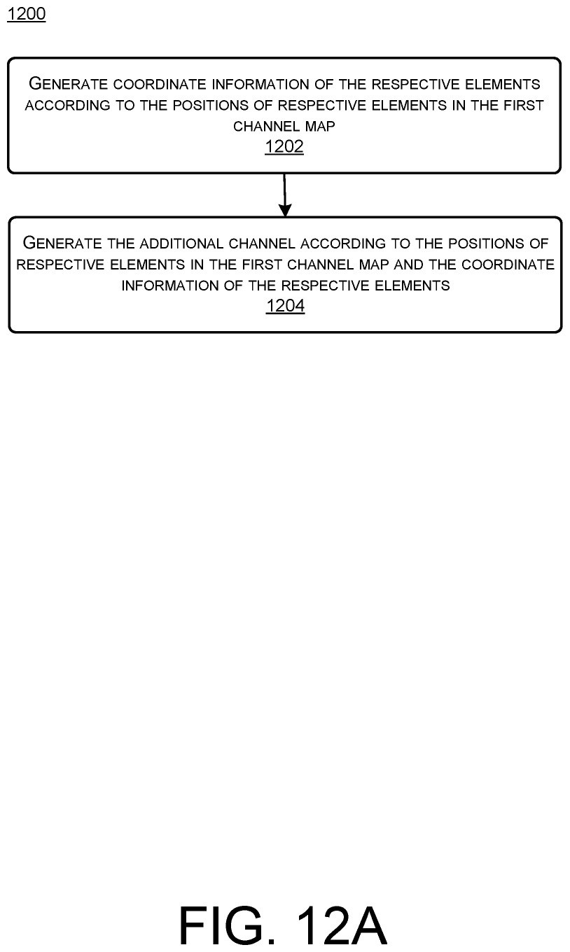

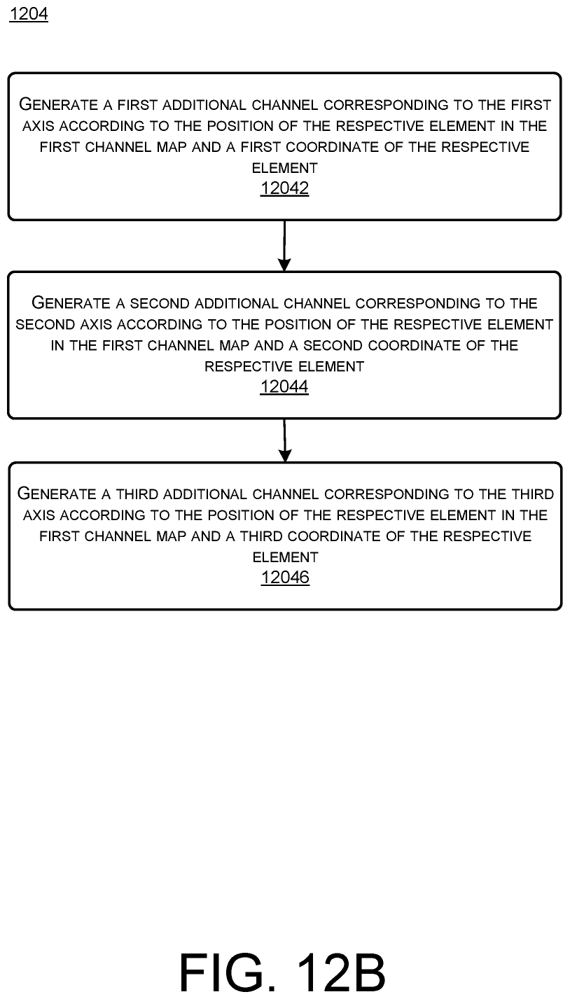

FIG. 12A and FIG. 12B are schematic flowcharts of a generating process 1200 of additional channels provided by an example embodiment of the present disclosure. As shown in FIG. 12A, the generating process 1200 includes the following steps:

1202. Generating coordinate information of the respective elements according to the positions of respective elements in the first channel map.

1204. Generating the additional channel according to the positions of respective elements in the first channel map and the coordinate information of the respective elements.

In the above 1202, when the first channel map is two-dimensional, the elements can be conceived as pixels. When the first channel map is three-dimensional, the elements can be conceived as voxels.

The first channel map may be an array. When the first channel map is two-dimensional, the first channel map is a two-dimensional array, and the position of the respective element in the first channel map is also the row and column positions (X, Y) of the perspective element in the two-dimensional array, where X refers to the row position, and Y refers to the column position. When the first channel map is three-dimensional, the first channel map is a three-dimensional array, and the position of the respective element in the first channel map is also the row, column, and page positions (X, Y, Z) in the three-dimensional array, where X refers to the row position, Y refers to the column position, and Z refers to the page position.

According to the position of the respective element in the first channel map, the step of generating coordinate information of the respective element may be implemented by one of the following methods:

Method 1: The position information of the respective element is directly used as the coordinate information of the respective element.

Hereinafter, the first channel map which is a 2*2*2 three-dimensional array is taken as an example for illustration. The 2*2*2 three-dimensional array includes eight elements: elements A, B, C, D, E, F, G, and H. The position information of element A is (2, 2, 1), and the coordinate information of element A is (2, 2, 1).

Method 2: Normalization processing (that is, standardization processing) is performed on the position information of the respective element in the first channel map to obtain coordinate information of the respective element.

In the finally obtained coordinate information of the respective element, the coordinate values are between −1 and 1. The normalization processing on the position information of the respective element is helpful for subsequent feature extraction.

Following the above example, the position information (2, 2, 1) of element A is standardized, that is, the page position, row position, and column position are divided by 2 to obtain (1, 1, 0.5). That is, the coordinate information of element A is (1, 1, 0.5).

In the above 1204, according to the position of the respective element in the first channel map, the coordinate information of the respective element is filled in at the corresponding position in the additional channel.

Taking the image to be processed which is a three-dimensional as an example, the first channel map is a three-dimensional channel map, and the coordinate information includes a first coordinate on a first axis, a second coordinate on a second axis, and a third coordinate on a third axis, where the first axis, the second axis, and the third axis intersect one with another. Referring to FIG. 12B, in the above 1204, “generating the additional channel according to the position of the respective element in the first channel map and the coordinate information of respective element” may be implemented by the following steps:

12042. Generating a first additional channel corresponding to the first axis according to the position of the respective element in the first channel map and a first coordinate of the respective element.

12044. Generating a second additional channel corresponding to the second axis according to the position of the respective element in the first channel map and a second coordinate of the respective element.

12046. Generating a third additional channel corresponding to the third axis according to the position of the respective element in the first channel map and a third coordinate of the respective element.

In the above 12042, the first coordinate of the respective element is filled in at the corresponding position in the first additional channel corresponding to the first axis according to the position of the respective element in the first channel map. Following the above example, the first channel map is a 2*2*2 three-dimensional array, and the first additional channel is also a 2*2*2 three-dimensional array. The position information of element A (2, 2, 1), and the coordinate information of element A information is (1, 1, 0.5). Assuming that the first axis is the x-axis, the first coordinate of element A, i.e., 1, is filled in at the position (2, 2, 1) in the first additional channel.

In the above 12044, according to the position of the respective element in the first channel map, the second coordinate of the respective element is filled in at the corresponding position in the second additional channel corresponding to the second axis. Following the above example, assuming that the second axis is the y-axis, the second coordinate of element A, i.e., 1, is filled in at the position (2, 2, 1) in the second additional channel.

In the above 12046, according to the position of the respective element in the first channel map, the third coordinate of the respective element is filled in at the corresponding position in the third additional channel corresponding to the third axis. Following the above example, assuming the third axis is the z-axis, the third coordinate of element A, i.e., 0.5, is filled in at the position (2, 2, 1) in the third additional channel.

In an example, the above step 1202 and step 1204 may be implemented by the above first network layer.

In an implementable solution, the above step of “determining the output data of the first network layer based on the input data of the additional channel” includes: performing feature extraction on the input data to which the additional channel is added, to obtain a third feature map as the output data of the first network layer.

In another implementable solution, the above step of “determining the output data of the first network layer based on the input data of the additional channel” includes: taking the input data to which the additional channel is added as the output data of the first network layer. In the example embodiment, the first network layer only performs the step of adding the additional channel. In implementations, the multiple network layers further include a convolution layer connected to the first network layer, and the convolution layer is configured to perform feature extraction on the output data of the first network layer to obtain the third feature map.

Further, the multiple network layers include an input layer and an output layer. The first network layer is located between the input layer and the output layer. The positions and the number of the first network layers in the neural network system can be set according to actual needs, and are not limited in the example embodiments of the present disclosure.

Through research, the inventor found that if the first network layer is provided at a position close to the front (i.e., being set close to the upstream) in the neural network system, as feature extraction is continuously performed subsequently, the coordinate information will be weakened, resulting in reduced improvement in the accuracy of segmentation. If the first network layer is provided prior to the output layer of the neural network system and connected to the output layer, the segmentation accuracy can be greatly improved.

In an example embodiment, only one first network layer is provided in the neural network system, and the first network layer is connected to the output layer.

What needs to be added is that the above multiple network layers may further include a convolution layer, a down-sampling layer, an up-sampling layer, and the like. For processing procedures of other network layers other than the first network layer among the multiple network layers, reference may be made to the conventional techniques, and is not limited herein.

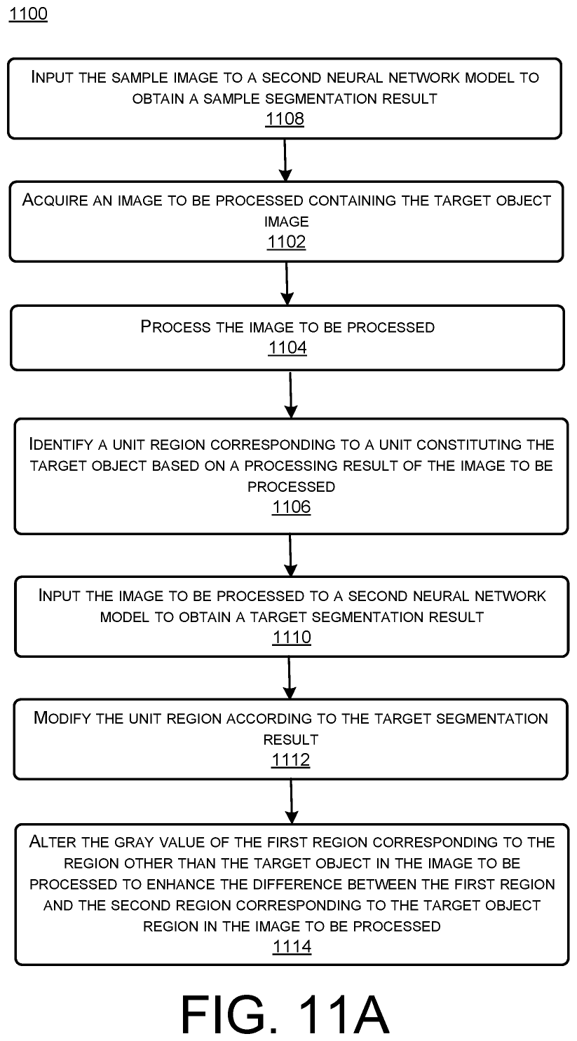

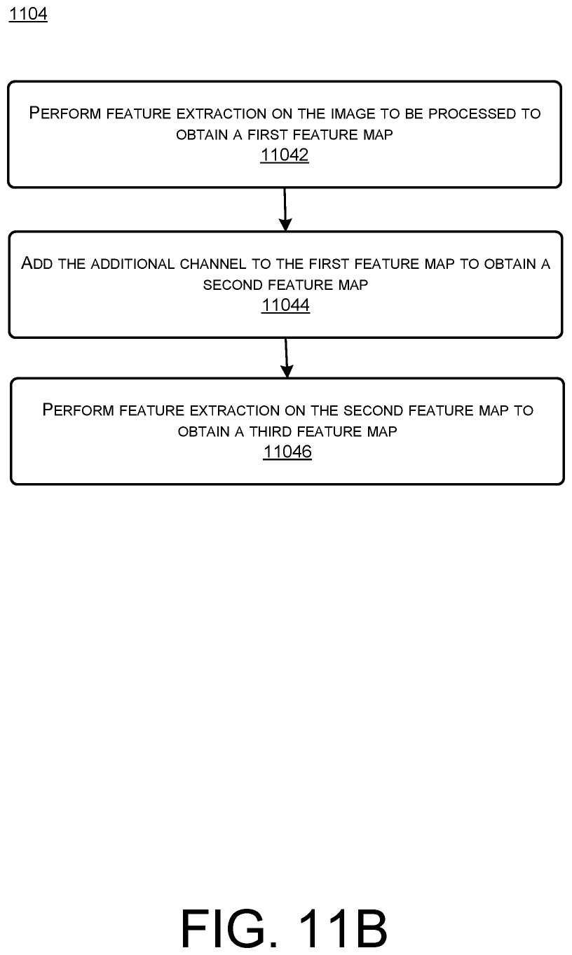

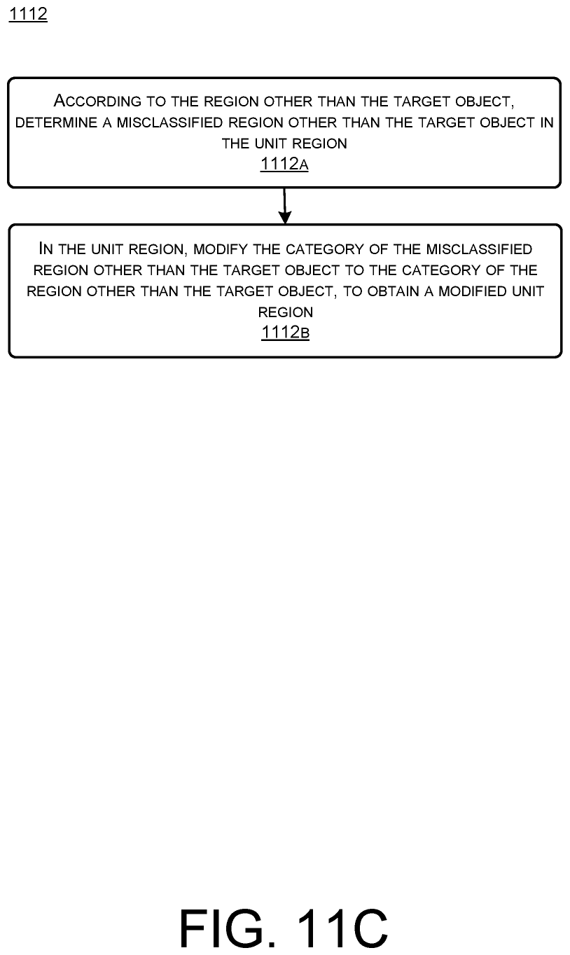

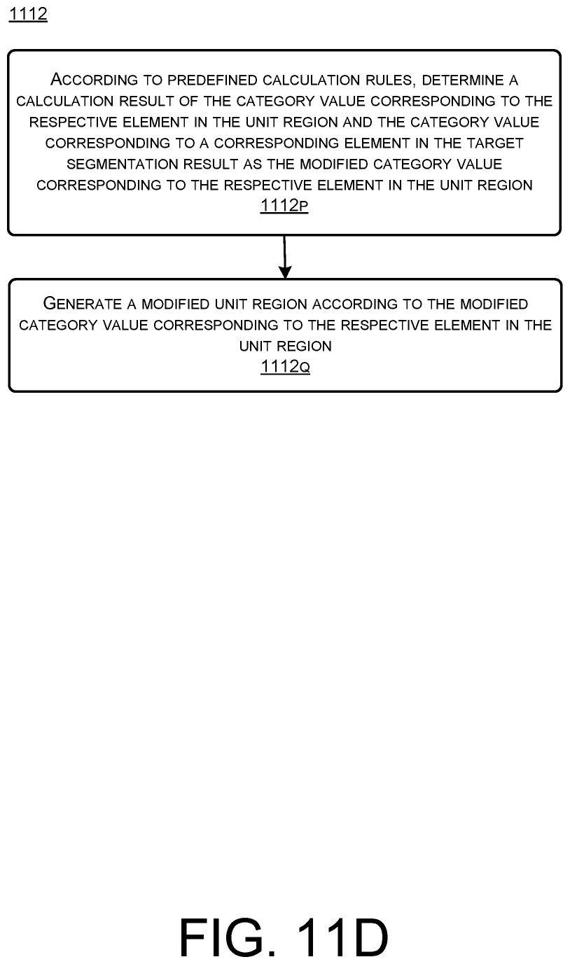

FIG. 11A is a schematic flowchart of a segmentation method 1100 provided by an example embodiment of the present disclosure. As shown in FIG. 11A, the method 1100 includes:

1102. Acquiring an image to be processed containing the target object image. 1104. Processing the image to be processed, where the processing procedure includes an operation of adding an additional channel with coordinate information.

1106. Identifying a unit region corresponding to a unit constituting the target object based on a processing result of the image to be processed.

In the above 1102, in actual applications, the image to be processed may be an image containing a biological body or an biological organ image acquired by a medical device (such as a CT device or an X-ray device), or an image containing an internal structure image of a device acquired by an industrial device (such as an industrial CT device), and the like, which is not limited in the example embodiments of the present disclosure. The target object may be an image of an object of interest in the image to be processed, for example, a device image with multiple constituent units, an animal body with multiple constituent units, an animal body organ with multiple constituent units, and the like.

In an implementable solution, in the above 1104, an additional channel with coordinate information may be directly added to the image to be processed. For the generating process of the additional channel, reference may be made to corresponding content in the above various example embodiments, and details are not repeated herein.

In another implementable solution, referring to FIG. 11B, in the above 1104, “processing the image to be processed” may be implemented by the following steps:

11042. Performing feature extraction on the image to be processed to obtain a first feature map.

11044. Adding the additional channel to the first feature map to obtain a second feature map.

In the above 11042, the first feature map may be obtained through performing the feature extraction on the image to be processed by at least one network layer in the first neural network model.

For the generating process of the additional channel in the above 11044, reference may be made to corresponding content in the above various example embodiments, and details are not repeated herein.

In the above 1106, the unit region corresponding to the unit constituting the target object is identified based on the processing result of the image to be processed.

In the technical solution provided by the example embodiment of the present disclosure, when the unit region corresponding to respective unit constituting the target object is divided, since the relative position distribution of the respective units constituting the target object is relatively fixed, the introduction of coordinate information may enable the neural network system to learn the coordinate features and global information, which play the role of coordinate guidance, effectively reducing the misclassification, and improving the accuracy of segmentation.

Further, the “processing the image to be processed” in the above 1104 may further include the following steps:

110046. Performing feature extraction on the second feature map to obtain a third feature map.

For example, the feature extraction is performed on the second feature map through a convolution operation.

In an example, the above step of adding the additional channel and the step of performing feature extraction on the second feature map to obtain the third feature map may be implemented by the first network layer in the first neural network model.

The training process of the first neural network model in the example embodiments of the present disclosure is as follows. A sample image containing a sample object image is input to the first neural network model. A predicted unit region corresponding to a unit constituting the sample object is identified, where the first network layer in the first neural network model is configured to add an additional channel with coordinate information to the input data input to the first network layer, and to determine the output data of the first network layer based on the input data to which the additional channel is added. Parameter optimization is performed on the first neural network model according to the predicted unit region and a desired unit region of the sample image, where the first neural network model is configured to identify the constituent region corresponding to the unit constituting the target object.

Further, the step of “performing parameter optimization on the first neural network model according to the predicted unit region and the desired unit region of the sample image” may include: executing Dice coefficient loss function to obtain a loss value using the predicted unit region and the desired unit region of the sample image as an input of the Dice coefficient loss function; and performing parameter optimization on the first neural network model according to the loss value. If the loss value is greater than or equal to the loss threshold value, the parameter optimization is performed on the first neural network model according to the loss value. If the loss value is less than the loss threshold value, the training is stopped and the obtained first neural network model can be put into use.

Further, prior to inputting the sample image to the first neural network model, the method further includes:

1108. Inputting the sample image to a second neural network model to obtain a sample segmentation result, where the sample segmentation result includes the identified sample object region and region other than the sample object; and altering the gray value of the first region corresponding to the region other than the sample object in the sample image to enhance the difference between the first region and the second region corresponding to the sample object region in the sample image. As such, the first neural network model may be facilitated to learn the features of the respective units. Also, the network convergence may be speeded up.

Further, the first neural network model includes an input layer and an output layer. The first network layer is located between the input layer and the output layer, and is connected to the output layer. As such, the accuracy of segmentation can be greatly improved.

In order to avoid the situation where the region other than the target object are misclassified in the segmentation result of the constituent unit of the target object, the above method may further include:

1110. Inputting the image to be processed to a second neural network model to obtain a target segmentation result.

The target segmentation result includes the identified target object region.

1112. Modifying the unit region according to the target segmentation result.

In the above 1110, the target object region is the region occupied by the identified target object. The above target segmentation result may also include the region other than the target object. The region other than the target object is the region other than the region occupied by the target object in the first image.

The second neural network model may use U-net as basic network architecture. The second neural network model may be obtained by training in advance through a public data set with object contour annotations. The second neural network model can also perform smoothing processing on the predicted object contour to obtain a complete and continuous object region. For the training process of the second neural network model, reference may be made to the conventional techniques, which is not described in detail herein.

What needs to be added is that when the second neural network model can only process two-dimensional images and the image to be processed is a three-dimensional image, the image to be processed may be divide into multiple two-dimensional images and input to the second neural network respectively, to obtain multiple two-dimensional target segmentation results; and then stitching the multiple two-dimensional target segmentation results into a three-dimensional target segmentation result for subsequent use.

In an implementable solution, referring to FIG. 11C, in the above 1112 “modifying the unit region according to the target segmentation result” may be implemented by the following steps:

1112a. According to the region other than the target object, determining a misclassified region other than the target object in the unit region.

1112b. In the unit region, modifying the category of the misclassified region other than the target object to the category of the region other than the target object, to obtain a modified unit region.

In the above 1112b, the misclassified region other than the target object refers to the region of the identified unit region where a region originally belonging to a region category other than the target object is predicted as a target object unit category.

The intersection of the identified unit region and the region other than the target object in the target segmentation result is determined as the misclassified region other than the target object.

In another implementable solution, the category value corresponding to the respective element in the target object region in the target segmentation result is assigned the first value (the first value represents the target object region category). The category value corresponding to the respective element in the region other than the target object is assigned the second value (the second value represents the category of the region other than the target object, i.e., the background category). Referring to FIG. 11D, the above 1112 “modifying the unit region according to the target segmentation result” may include:

1112p. According to predefined calculation rules, determining a calculation result of the category value corresponding to the respective element in the unit region and the category value corresponding to a corresponding element in the target segmentation result as the modified category value corresponding to the respective element in the unit region.

1112q. Generating a modified unit region according to the modified category value corresponding to the respective element in the unit region.

The calculation rules include: the calculation result of the first value and a third value is the third value; and the calculation result of the second value and a fourth value is the second value.

The position information of the respective element in the constituent unit segmentation result is the same as the position information of the corresponding element of the respective element in the target segmentation result.

For example, the position of element A in the identified unit region is (150, 162, 32), and the category value corresponding to element A is 1. The position of element A′ in the target segmentation result is also (150, 162, 32), and the category value corresponding to element A′ is 0. Element A′ is the corresponding element of element A. The category value 1 corresponding to element A is multiplied by the category value 0 corresponding to element A′, and the modified category value corresponding to element A is obtained to be 0. As presented, the category value of element A in the constituent unit segmentation result is predicted mistakenly. Element A should belong to the region category other than the target object. As such, the situation of misclassification other than the target object in the subsequent constituent unit segmentation can be effectively modified.

In the example embodiment, with the assistance of the second neural network model, the situation of misclassification other than the target object can be reduced.

Further, the target segmentation result includes the identified target object region and the region other than the target object. Prior to the step of processing the image to be processed to add the additional channel with coordinate information therein to obtain the second feature map, the above method may further include:

1114. Altering the gray value of the first region corresponding to the region other than the target object in the image to be processed to enhance the difference between the first region and the second region corresponding to the target object region in the image to be processed.

Taking the target object which is the lung as an example, in the image to be processed, the gray value of the first region is uniformly set to a set value greater than a first preset threshold.

In the example embodiment, by enhancing the difference between the target object region and the region other than the target object in the image to be processed, subsequent neural networks may be facilitated to learn the features of the respective units of the target object, improving the accuracy.

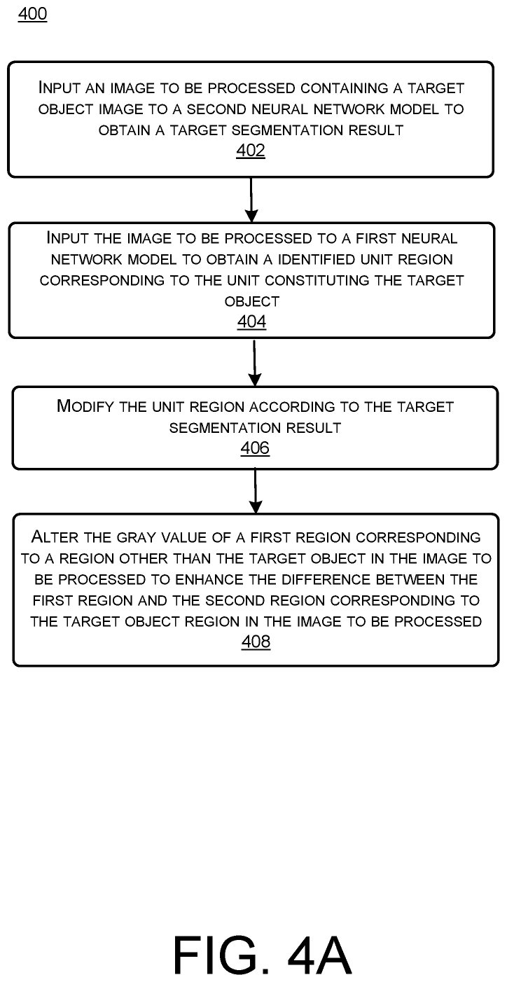

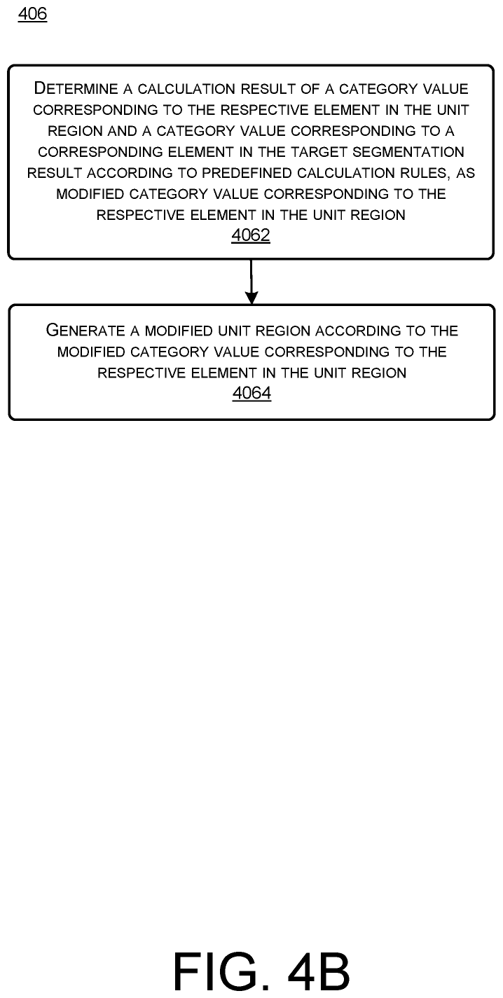

FIG. 4A illustrates a schematic flowchart of a segmentation method 400 provided by another example embodiment of the present disclosure. As shown in FIG. 4A, the method 400 includes:

402. Inputting an image to be processed containing a target object image to a second neural network model to obtain a target segmentation result.

The target segmentation result includes the identified target object region.

404. Inputting the image to be processed to a first neural network model to obtain an identified unit region corresponding to the unit constituting the target object.

406. Modifying the unit region according to the target segmentation result.

In the above 402, in actual applications, the image to be processed may be an image containing a biological body or biological organ image acquired by a medical device (such as a CT device or an X-ray device), or an image containing an internal structure image of a device acquired by an industrial device (such as an industrial CT device), and the like, which are not limited in the example embodiments of the present disclosure. The target object may be an image of the object of interest in the image to be processed.

The target object region is the region occupied by the identified target object. The target segmentation result may further include a region other than the target object, and the region other than the target object is a region other than the region occupied by the target object in the image to be processed.

For the training process of the second neural network model, reference may be made to corresponding content in the above various example embodiments, and details are not repeated herein.

In the above 404, for the first neural network model, may refer to a deep neural network model, a recurrent neural network model, and the like. The present disclosure does not limit the type of the first neural network model.

In order to improve the segmentation accuracy of the unit region, the first neural network model may be a neural network model based on coordinate guidance. In implementations, the first neural network model includes a first network layer. The first network layer is configured to add an additional channel with coordinate information to the input data input to the first network layer, and to determine the output data of the first network layer based on the input data after the additional channel is added thereto.

For the implementation of adding the additional channel with coordinate information to the input data, reference may be made to corresponding content in the above various example embodiments, and details are not repeated herein.

In an implementable solution, the step of determining the output data of the first network layer based on the input data after the additional channel is added thereto may be: performing feature extraction on the input data after the additional channel is added thereto to obtain the third feature map. That is, the first network layer not only performs the step of adding an additional channel, but also performs an operation of performing feature extraction on the input data after the additional channel is added thereto.

In actual applications, the above first neural network model may be obtained by improving the convolutional neural network (CNN) model, that is, replacing respective convolutional layer in at least one convolutional layer in the convolutional neural network model with the first network layer provided in the example embodiments of the present disclosure. The convolutional neural network may be a fully convolutional neural network model. In an implementable solution, the fully convolutional neural network model may use v-net as basic network architecture.

For implementations of the above 406, reference may be made to corresponding content in the above various example embodiments, and details are not repeated herein.

In the example embodiment, with the assistance of the second neural network model, the situation of misclassification of the region other than the target object may be reduced.

In an implementable solution, the target segmentation result further includes the identified region other than the target object. The category value corresponding to the respective element in the target object region in the target segmentation result is assigned the first value. The category value corresponding to the respective element in the region other than the target object is assigned the second value.

Referring to FIG. 4B, in the above 406, “modifying the unit region according to the target segmentation result” may be implemented by the following steps:

4062. Determining a calculation result of a category value corresponding to the respective element in the unit region and a category value corresponding to a corresponding element in the target segmentation result according to predefined calculation rules, as modified category value corresponding to the respective element in the unit region.

4064. Generating a modified unit region according to the modified category value corresponding to the respective element in the unit region.

The calculation rules include: the calculation result of the first value and a third value is the third value; and the calculation result of the second value and a fourth value is the second value.

Further, prior to inputting the image to be processed to the first neural network model, the above method 400 may further include:

408. Altering the gray value of a first region corresponding to a region other than the target object in the image to be processed to enhance the difference between the first region and the second region corresponding to the target object region in the image to be processed.

For implementations of the above 408, reference may be made to corresponding content in the above various example embodiments, and details are not repeated herein. The altered image to be processed is input to the first neural network model.

In the example embodiment, by enhancing the difference between the target object region and the region other than the target object in the image to be processed, subsequent neural networks may be facilitated to learn the features of respective constituent unit of the target object, improving the accuracy.

Further, the first neural network model further includes an input layer and an output layer. The first network layer is located between the input layer and the output layer.

The positions and the number of the first network layers in the first neural network model can be set according to actual needs, and are not limited in the example embodiments of the present disclosure.

Through research, the inventor found that if the first network layer is provided at a position close to the front in the first neural network model, as feature extraction (such as convolution) is continuously performed subsequently, the coordinate information will be weakened, resulting in reduced improvement in the accuracy of constituent unit segmentation. If the first network layer is provided prior to the output layer and connected to the output layer, the accuracy of unit region segmentation can be greatly improved.

In an example, only one first network layer is provided in the first neural network model, and the first network layer is connected to the output layer.

For the training process of the first neural network model in the example embodiments of the present disclosure, reference may be made to corresponding content in the above various example embodiments, and details are not repeated herein.

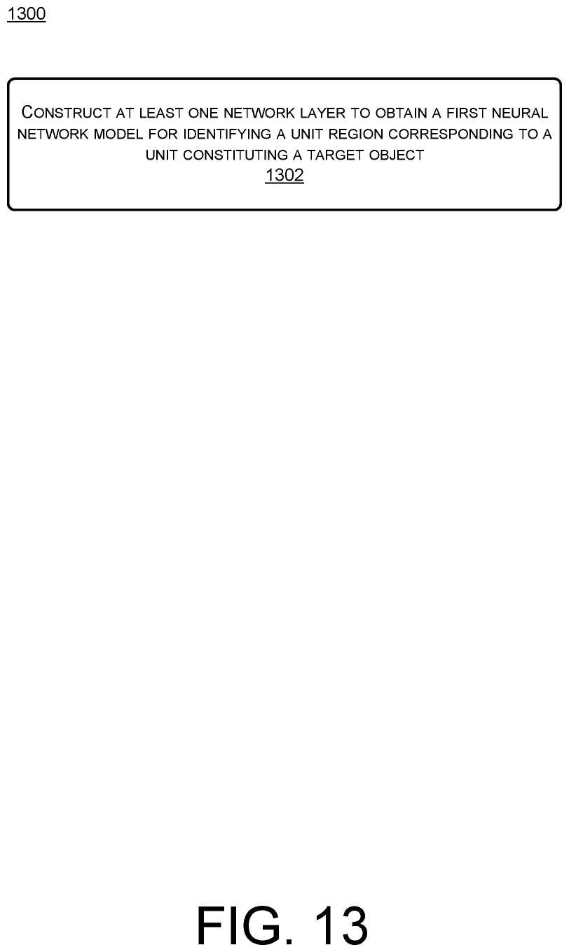

As shown in FIG. 13, a method 1300 for constructing the first neural network model is described hereinafter. The method 1300 may include 1302 constructing at least one network layer to obtain a first neural network model for identifying a unit region corresponding to a unit constituting a target object; where the at least one network layer includes at least one first network layer; and the first network layer is configured to add an additional channel with coordinate information to input data input to the first network layer, and to determine the output data of the first network layer based on the input data to which the additional channel is added.

What needs to be noted here is that for details that are not described in the steps of the method provided in the example embodiments of the present disclosure, reference may be made to the corresponding contents in the above example embodiments, and details are not repeated herein. In addition, the methods provided in the example embodiments of the present disclosure may include other or all steps in the above various example embodiments in addition to the above steps. Reference may be made to the corresponding content of the above example embodiments, and details are not repeated herein.

The technical solutions provided by the various example embodiments of the present disclosure may be applicable to various application scenarios, such as a scenario in which medical images or industrial CT acquisition flaw detection images are segmented. In the following various example embodiments, a scenario in which a medical image is segmented is taken as an example to describe the present solution. Hereinafter, a model construction method is introduced. The method may include:

Constructing at least one network layer to obtain a first neural network model for lung lobe segmentation.

The at least one network layer includes at least one first network layer; where the first network layer is configured to add an additional channel with coordinate information to input data input to the first network layer, and to determine the output data of the first network layer based on the input data after the additional channel is added thereto.

Processing of other network layers other than the first network layer in at least one network in the first neural network model is the same as that in the conventional techniques, and details are not repeated herein.

In the technical solutions provided by the example embodiments of the present disclosure, the first network layer is designed in the first neural network model for lung lobe segmentation. The first network layer adds the additional channel with coordinate information to the input data input to the first network layer, and determines the output data of the first network layer based on the input data after the additional channel is added thereto. The relative position distribution of the five lung lobes in the lung image is relatively fixed. The introduction of coordinate information may enable the neural network model to learn the coordinate features and global information, which play the role of coordinate guidance, effectively reducing the misclassification within the lung, and improving the accuracy of lung lobe segmentation.

In an example, the image to be processed containing the lung image may be a computed tomography (CT) image of the chest.

The positions and the number of the first network layers in the first neural network model can be set according to actual needs, and are not limited in the example embodiments of the present disclosure.

Through research, the inventor found that if the first network layer is provided to a position close to the upstream in the first neural network model, as the feature extraction is continuously performed subsequently, the coordinate information will be weakened, resulting in reduced improvement in the accuracy of lung lobe segmentation. If only one first network layer is designed, and the first network layer is provided prior to the output layer and connected to the output layer, the accuracy of lung lobe segmentation can be greatly improved. In implementations, the number of the first network layers in the at least one network layer is one. The at least one network layer includes an input layer and an output layer. The first network layer is located in the input layer and the output layer, and the first network layer is connected to the output layer.

Further, the step of determining the output data of the first network layer based on the input data after the additional channel is added thereto may include: performing feature extraction on the input data after the additional channel is added thereto to obtain a third feature map. That is, the first network layer not only can perform the step of adding an additional channel, but also can perform an operation of performing feature extraction. In actual applications, the above first neural network model may be obtained by improving the convolutional neural network (CNN) model, that is, at least one convolutional layer in the convolutional neural network model is replaced by the first network layer provided by the example embodiments of the present disclosure. The convolutional neural network may be a fully convolutional neural network model. In an implementable solution, the full-convolutional neural network model may use v-net as basic network architecture.

For the processing performed by the above first network layer, reference may be made to corresponding content in the above various example embodiments, and details are not repeated herein.

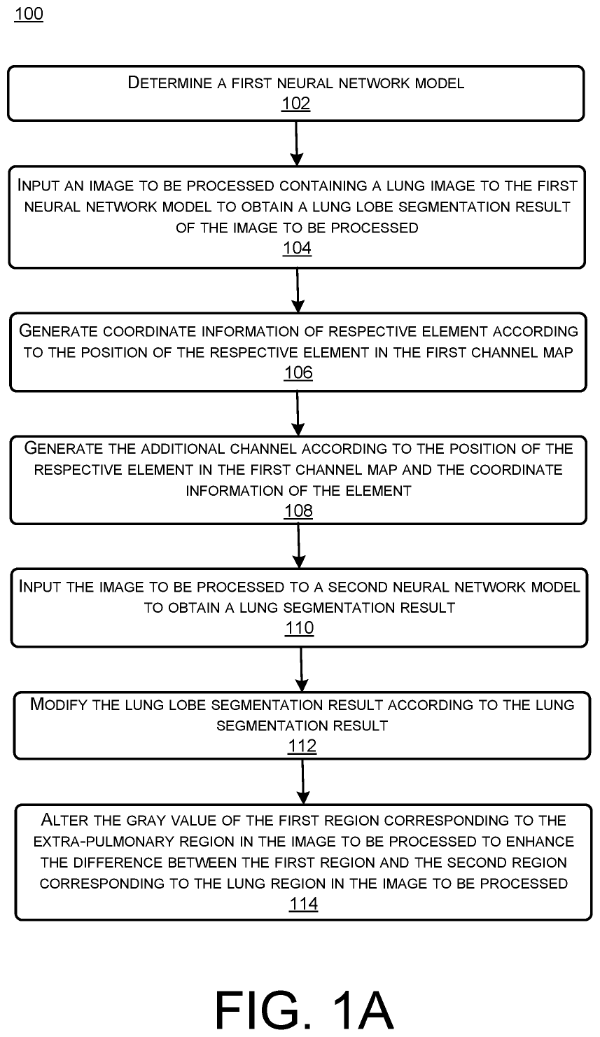

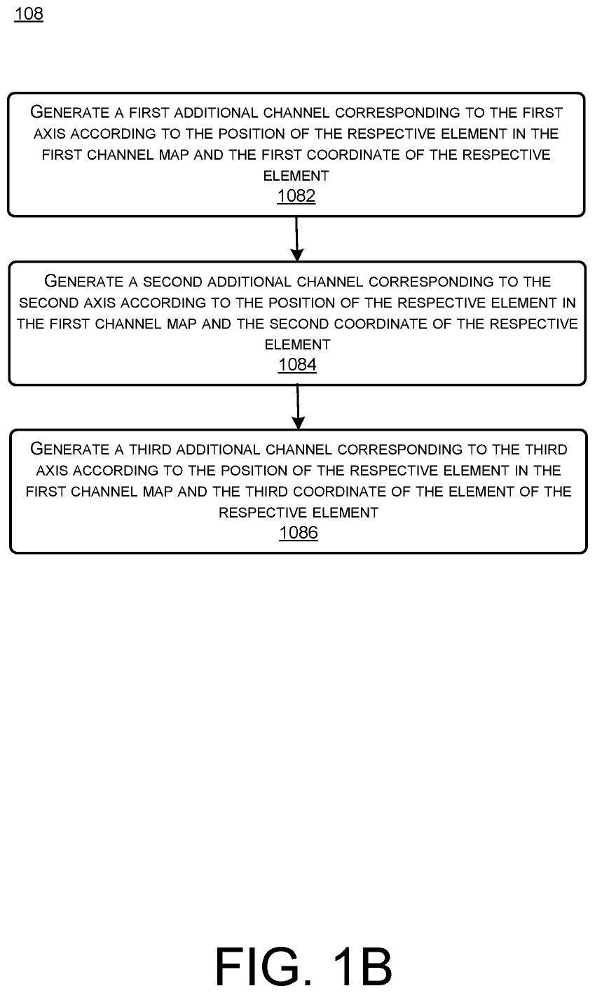

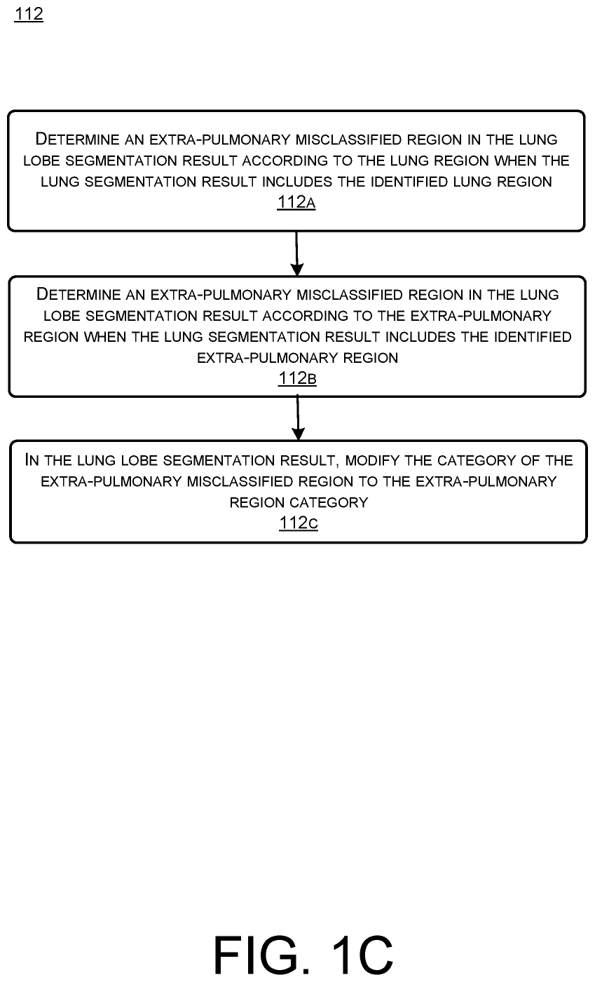

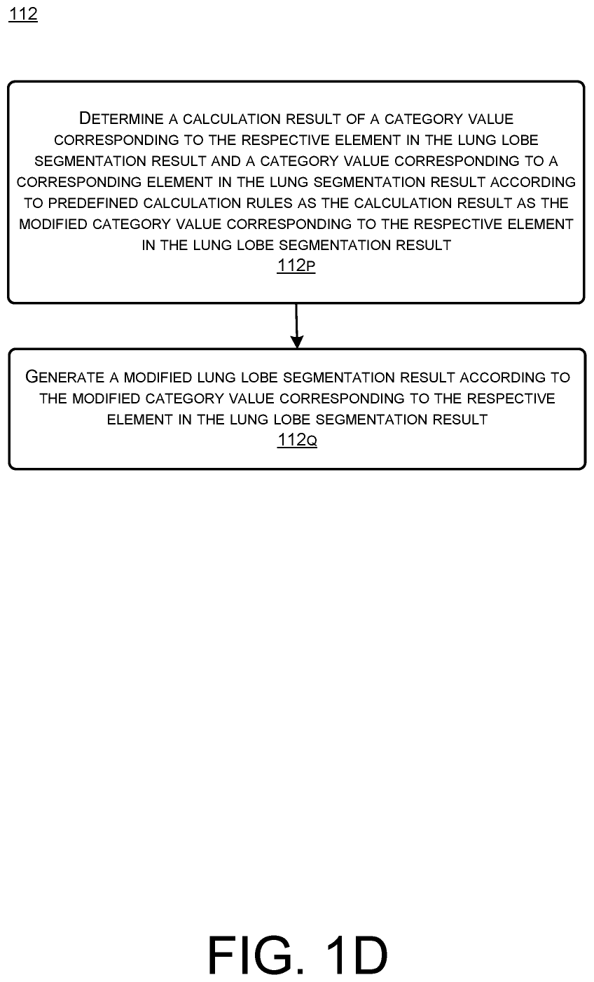

FIG. 1A is a schematic flowchart of a lung lobe segmentation method 100 provided by an example embodiment of the present disclosure. As shown in FIG. 1A, the method 100 includes:

102. Determining a first neural network model.

104. Inputting an image to be processed containing a lung image to the first neural network model to obtain a lung lobe segmentation result of the image to be processed.

The first network layer in the first neural network model is configured to add an additional channel with coordinate information to input data input to the first network layer, and to determine output data of the first network layer based on the input after adding the additional channel data.

In the above 104, an image to be processed containing a lung image may be input to an input layer in a first neural network model. The lung lobe segmentation result may include the identified lobe regions of multiple lobes (usually five) and the background region (i.e., the extra-pulmonary region).

In actual applications, before the image to be processed is input to the input layer, the image to be processed may be pre-processed to meet the size requirement of the input image for the first neural network model, which is convenient for subsequent prediction. For example, the image to be processed is a three-dimensional image, and the size can be pre-processed to be 128*256*256 through bilinear interpolation, where 128 represents the depth of the pre-processed image, and two 256 respectively represent the width and the height of the pre-processed image to be processed.

The input data input to the first network layer may be the image to be processed or a first feature map obtained after feature extraction is performed on the image to be processed. The first network layer may be the input layer, the output layer, or any layer between the input layer and the output layer of the first neural network model, which is not limited in the example embodiment.

In implementations, the input data may include at least one channel map. When the input data includes two or more channel maps, the input data may be referred to as a multi-channel feature map. When the input data includes only one channel, the input data may be referred to as a single-channel feature map.

For the implementation of adding the additional channel to the input data and the generating process of the above additional channel, reference may be made to corresponding content in the above various example embodiments, and details are not repeated herein.

In the technical solutions provided in the example embodiments of the present disclosure, the first network layer is designed in the first neural network model for lung lobe segmentation, and the first network layer adds an additional channel having coordinate information to the input data input to the first network layer, and determines the output data of the first network layer based on the input data after the additional channel is added thereto. The relative position distribution of the five lung lobes in the lung image is relatively fixed. The introduction of coordinate information may enable the neural network model to learn the coordinate features and global information, which play the role of coordinate guidance, effectively reducing the misclassification within the lung, and improving the accuracy of lung lobe segmentation.

In an implementable solution, the input data is a multi-channel feature map, that is, the input data includes at least two channel maps. The input data includes a first channel map. The first channel map refers to any one of the at least two channel maps. The above method 100 may further include:

106. Generating coordinate information of respective element according to the position of the respective element in the first channel map.

108. Generating the additional channel according to the position of the respective element in the first channel map and the coordinate information of the element.

For implementations of the above 106 and 108, reference may be made to corresponding content in the above various example embodiments, and details are not repeated herein. The step of generating coordinate information of the respective element according to the position of the respective element in the first channel map may be implemented by the following method: performing normalization processing (i.e., standardization processing) on the position information of the respective element in the first channel map to obtain coordinate information of the respective element. For details of the normalization processing, reference may be made to corresponding content in the above various example embodiments, and details are not repeated herein.

What needs to be added is that the above steps 106 and 108 may be implemented by the above first network layer.

Taking the image to be processed which is a three-dimensional image as an example, the first channel map is a three-dimensional channel map. The coordinate information includes a first coordinate on a first axis, a second coordinate on a second axis, and a third coordinate on a third axis, where the first axis, the second axis, and the third axis intersect one with another.

Referring to FIG. 1B, in the above 108, “generating the additional channel according to the position of the respective element in the first channel map and the coordinate information of respective element” may be implemented by the following steps:

1082. Generating a first additional channel corresponding to the first axis according to the position of the respective element in the first channel map and the first coordinate of the respective element.

1084. Generating a second additional channel corresponding to the second axis according to the position of the respective element in the first channel map and the second coordinate of the respective element.

1086. Generating a third additional channel corresponding to the third axis according to the position of the respective element in the first channel map and the third coordinate of the element of the respective element.

For implementations of the above 1082, 1084, and 1086, reference may be made to corresponding content in the above various example embodiments, and details are not repeated herein. In order to avoid the situation of extra-pulmonary misclassification within the lung lobe segmentation result, the above method 100 may further include:

110. Inputting the image to be processed to a second neural network model to obtain a lung segmentation result.

The lung segmentation result includes the identified lung region and/or an extra-pulmonary region.

112. Modifying the lung lobe segmentation result according to the lung segmentation result.

In the above 110, the second neural network model may use U-net as basic network architecture, and the second neural network model may be obtained by training in advance through a public data set with lung contour annotations. The second neural network model can also perform smoothing processing on the predicted lung contour to obtain a complete and continuous lung region. As verified, the accuracy of lung segmentation in this part can reach more than 99.5%. For the training process of the second neural network model, reference may be made to the conventional techniques, which are not described in detail herein.

In the above 112, the lung lobe segmentation result is modified based on the identified lung region or extra-pulmonary region in the lung segmentation result. That is, by extracting the lung region or extra-pulmonary region, the extra-pulmonary misclassification in subsequent lung lobe segmentation can be effectively modified.

Referring to FIG. 1C, in an implementable solution, in the above 112, “modifying the lung lobe segmentation result according to the lung segmentation result”, may be implemented by the following steps:

112a. Determining an extra-pulmonary misclassified region in the lung lobe segmentation result according to the lung region when the lung segmentation result includes the identified lung region.

112b. Determining an extra-pulmonary misclassified region in the lung lobe segmentation result according to the extra-pulmonary region when the lung segmentation result includes the identified extra-pulmonary region.

112c. In the lung lobe segmentation result, modifying the category of the extra-pulmonary misclassified region to the extra-pulmonary region category.

In the above 112a and 112b, the extra-pulmonary misclassified region refers to a region originally belonging to the extra-pulmonary region category that is predicted to be a certain lung lobe category in the lung lobe segmentation result.

Determining the intersection of the total region composed of five lung lobe regions in the lung lobe segmentation result with the extra-pulmonary region in the lung segmentation result as the extra-pulmonary misclassified region; or excluding the lung region in the lung segmentation result from the total region composed of five lung lobe regions in the lung lobe segmentation result, with the remaining region being the extra-pulmonary misclassified region.

In the above 112c, in the lung lobe segmentation result, the category of the extra-pulmonary misclassified region is modified to the extra-pulmonary region category.

In another implementable solution, the category value corresponding to the respective element in the lung region in the lung segmentation result is assigned the first value (the first value represents the lung region category). The category value corresponding to the respective element in the extra-pulmonary region is assigned the second value (the second value represents the extra-pulmonary region category, i.e., the background category). The lung lobe segmentation result includes five lobe regions (upper left lobe, left lower lobe, right upper lung lobe, right middle lung lobe, and lower right lung lobe). The category value corresponding to the respective element in respective lung lobe region can be assigned according to actual needs, and the example embodiments of the present disclosure are not limited herein. For example, the category value corresponding to the respective element in the upper left lung lobe region is 1. The category value corresponding to the respective element in the lower left lung lobe is 2. The category value corresponding to the respective element in the upper right lung lobe is 3. The category value corresponding to the respective element in the right middle lung lobe is 4. The category value corresponding to the respective element in the right lower lung lobe is 5.

Referring to FIG. 1D, in the above 112, “modifying the lung lobe segmentation result according to the lung segmentation result” includes:

112p. Determining a calculation result of a category value corresponding to the respective element in the lung lobe segmentation result and a category value corresponding to a corresponding element in the lung segmentation result according to predefined calculation rules as the calculation result as the modified category value corresponding to the respective element in the lung lobe segmentation result.

112q. Generating a modified lung lobe segmentation result according to the modified category value corresponding to the respective element in the lung lobe segmentation result.

The calculation rules include: the calculation result of the first value and a third value is the third value; and the calculation result of the second value and a fourth value is the second value.

The position information of the respective element in the lung lobe segmentation result is the same as the position information of the corresponding element of the respective element in the lung segmentation result.

For example, the position of element A in the lung lobe segmentation result is (150, 162, 32). The category value corresponding to element A is 1. The position of element A′ in the lung segmentation result is also (150, 162, 32). The category value corresponding to element A′ is 0. Element A′ is the corresponding element of element A. The category value 1 corresponding to element A is multiplied by the category value 0 corresponding to element A′, and the modified category value corresponding to element A is obtained to be 0. As presented, the category value of element A in the lung lobe segmentation result is predicted mistakenly. Element A should belong to the extra-pulmonary region category. As such, the extra-pulmonary misclassification in subsequent lung lobe segmentation can be effectively modified.

Further, in order to facilitate the subsequent neural network to learn the features of the lung lobes, the difference between the lung region and the extra-pulmonary region in the image to be processed may be enhanced before the image to be processed is input to the first neural network model. In implementations, the above method 100 may further include:

114. Altering the gray value of the first region corresponding to the extra-pulmonary region in the image to be processed to enhance the difference between the first region and the second region corresponding to the lung region in the image to be processed.

Generally, the gray value of the lung region is less than 1. The gray value of the extra-pulmonary region may be set to be greater than the first preset threshold. In implementations, in the image to be processed, the gray value of the first region is uniformly set to a set value greater than the first preset threshold.

The first preset threshold is a number greater than 1. The first preset threshold may be determined according to actual needs, which is not limited in the example embodiments of the present disclosure. The above set value may be 20, for example.

Further, the first neural network model further includes an input layer and an output layer. The first network layer is located between the input layer and the output layer.

The positions and the number of the first network layers in the first neural network model can be set according to actual needs, and are not limited in the example embodiments of the present disclosure.

Through research, the inventor found that if the first network layer is provided at a position close to the front in the first neural network model, as the feature extraction (such as convolution) is continuously performed subsequently, the coordinate information will be weakened, resulting in reduced improvement in the accuracy of lung lobe segmentation. If the first network layer is provided prior to the output layer and connected to the output layer, the accuracy of lung lobe segmentation can be greatly improved.

In an example embodiment, only one first network layer is provided in the first neural network model, and the first network layer is connected to the output layer.

Further, the step of determining the output data of the first network layer based on the input data after the additional channel is added thereto may include: performing feature extraction on the input data after the additional channel is added thereto to obtain a third feature map. That is, the first network layer not only can perform the step of the adding the additional channel, but also can perform the step of feature extraction.

In summary, because the relative position distribution of the five lung lobes in the lung image is relatively fixed, the introduction of coordinate information may enable the neural network model to learn the coordinate features, which play the role of coordinate guidance, effectively reducing the misclassification within the lung, and improving the accuracy of lung lobe segmentation. With the assistance of the second neural network model, the situation of extra-pulmonary misclassification can be reduced. By enhancing the difference between the gray value of the lung region and the gray value of the extra-pulmonary region in the image to be processed, subsequent neural networks may be facilitated to learn the features of the lung lobes, improving the accuracy, and speeding up network convergence.

What needs to be noted here is that for the content that is not described in detail in the steps of the method provided in the example embodiments of the present disclosure, reference may be made to the corresponding content in the above various example embodiments, and details are not repeated herein.

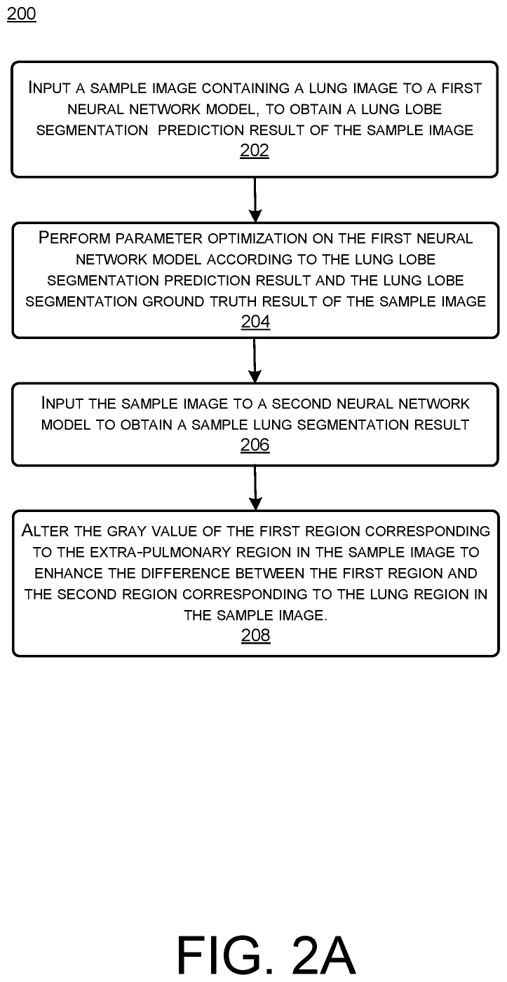

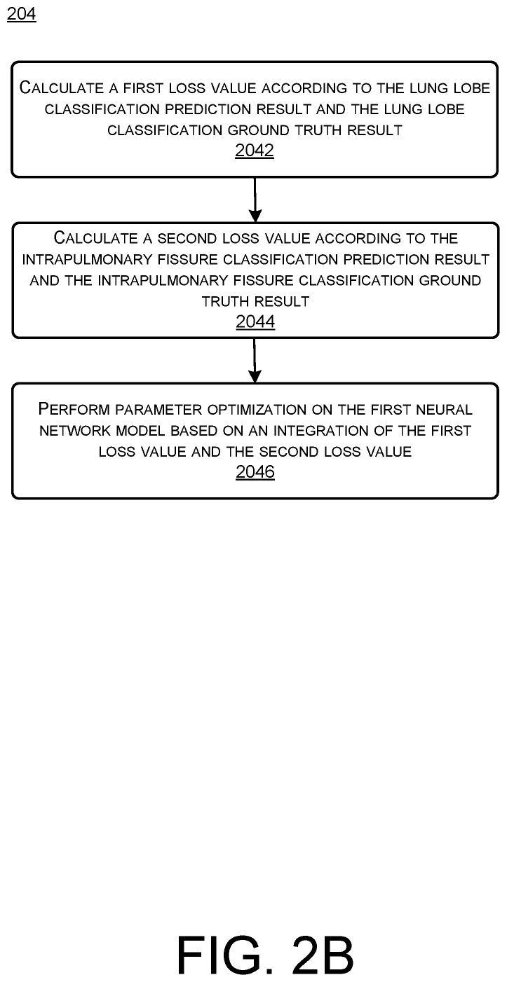

The training process of the first neural network model in the example embodiments of the present disclosure will be described hereinafter with reference to FIG. 2A. As shown in FIG. 2A, a model training method 200 includes:

202. Inputting a sample image containing a lung image to a first neural network model, to obtain a lung lobe segmentation prediction result of the sample image.

204. Performing parameter optimization on the first neural network model according to the lung lobe segmentation prediction result and the lung lobe segmentation ground truth result of the sample image.

The first neural network model is configured for lung lobe segmentation. The first network layer in the first neural network model is configured to add additional channel with coordinate information to the input data input to the first network layer, and to determine the output data of the first network layer based on the input data to which the additional channel is added.

In the above 202, for the processing procedure on the sample image by respective network layer in the first neural network, reference may be made to the processing procedure on the image to be processed containing the lung image by respective network layer in the first neural network in the above various example embodiments, and details are not repeated herein.

In the above 204, the difference value between the lung lobe segmentation prediction result and the lung lobe segmentation ground truth result, that is, the loss value, is calculated. The loss value between the lung lobe segmentation prediction result and the lung lobe segmentation ground truth result is greater than or equal to the first loss threshold, indicating that the network has not converged, and the parameter optimization needs to be performed on the first neural network model according to the loss value between the lung lobe segmentation prediction result and the lung lobe segmentation ground truth result. The loss value between the lung lobe segmentation prediction result and the lung lobe segmentation ground truth result is less than the first loss threshold, indicating that the network converges, and the parameter optimization can stop. That is, the model training stops, and the obtained first neural network model can be put into use.

During the model training process, a method of monitoring intrapulmonary fissures may be added to improve the accuracy of lung lobe segmentation. For the ground truth of the lung lobe category (i.e., the truth value of the lung lobe category) annotated in the sample image, the intrapulmonary fissures are obtained in advance through the erosion method. Then, Gaussian smoothing is used to obtain the ground truth of the intrapulmonary fissure category (i.e., the truth value of the intrapulmonary fissure category in the sample image). For the implementation of obtaining the intrapulmonary fissures through the corrosion method, reference may be made to conventional techniques, and details are not described herein. The above first neural network model learns the features of the lung lobes while learning the features of the intrapulmonary fissures. As verified, adding the method of monitoring intrapulmonary fissures may improve the accuracy of lung lobe segmentation.

In implementations, the lung lobe segmentation prediction result includes a lung lobe classification prediction result and an intrapulmonary fissure classification prediction result. The lung lobe segmentation ground truth result includes a lung lobe classification ground truth result and an intrapulmonary fissure classification ground truth result.

Referring to FIG. 2B, in the above 204, “performing parameter optimization on the first neural network model according to the lung lobe segmentation prediction result and the lung lobe segmentation ground truth result of the sample image” may be implemented by the following steps:

2042. Calculating a first loss value according to the lung lobe classification prediction result and the lung lobe classification ground truth result.

2044. Calculating a second loss value according to the intrapulmonary fissure classification prediction result and the intrapulmonary fissure classification ground truth result.

2046. Performing parameter optimization on the first neural network model based on an integration of the first loss value and the second loss value.

The first loss value and the second loss value may be calculated using an existing loss function.

In the above 2046, the following formula (1) may be used to calculate the total loss value D0 of the network:

D0=D1+λ*D2 (1)

where D1 is the first loss value, D2 is the second loss value, and the value of λ is (0, 1]. In an example, the value of λ is 1, that is, the total loss value is the sum of the first loss value and the second loss value.

In order to further improve the prediction accuracy of the first neural network model, the Dice coefficient loss function may be used to calculate the first loss value and the second loss value. Since the Dice coefficient loss function considers the intersection ratio (i.e., the overlapping part) between the lung lobe classification prediction result and the lung lobe classification ground truth result, the intersection ratio between the intrapulmonary fissure classification prediction result and the intrapulmonary fissure classification ground truth result, the consistency between the output result and the target result is considered, and the intersection ratio of the prediction and the ground truth results is maximized as much as possible. Moreover, with the Dice coefficient loss function, there is no need to deal with the classification imbalance problem.

In implementations, the above 2044 “calculating a first loss value according to the lung lobe classification prediction result and the lung lobe classification ground truth result” is: executing the first Dice coefficient loss function to obtain the first loss value, with the lung lobe classification prediction result and the lung lobe classification ground truth result as the input of first Dice coefficient loss function.

The formula of the first Dice coefficient loss function is as follows:

$${D}_{lobes} = {\sum }_{c} {D}_{lobes}^{c} = {\sum }_{c} ( - \frac{2 {\sum }_{i} {p}^{c} ( i ) {g}^{c} ( i )}{{\sum }_{i} {p}^{c} ( i ) + {\sum }_{i} {g}^{c} ( i ) + γ} ) \qquad ( 2 )$$

where g^{c}(i) is the lung lobe category truth value corresponding to the i-th element (i.e., the lung lobe category ground truth); p^{c}(i) is the lung lobe category to which the i-th element belongs predicted by the network; the value of c is the five category values 1, 2, 3, 4, 5 (that is, c only takes the category values corresponding to five lung lobes, and c does not take the background category value); γ is 1e−5; and D_{lobes}^{c} represents the loss value between the lung lobe classification prediction result and the lung lobe classification ground truth result of the lung lobe region where the lung lobe category ground truth corresponding to the respective element in the sample image is c.

What needs to be added here is that when the output layer classifies the respective element in the sample image and the image to be processed in the above various example embodiments, the output layer only selects one of the category values corresponding to the five lung lobes as the prediction value corresponding to the respective element. As such, the first neural network model can effectively avoid mistakenly classifying the lung lobe region into an extra-pulmonary region category (i.e., the background category), and improve the accuracy of segmentation.

In the above 2044, “calculating a second loss value based on the intrapulmonary fissure classification prediction result and the intrapulmonary fissure classification ground truth result” is: executing the second Dice coefficient loss function to obtain the second loss value, with the intrapulmonary fissure classification prediction result and the intrapulmonary fissure classification ground truth result as the input of the second Dice coefficient loss function.

The formula of the second Dice coefficient loss function (3) is as follows:

$${D}_{boundary} = - \frac{2 {\sum }_{i} {p}^{b} ( i ) {g}^{b} i}{{\sum }_{i} {p}^{b} ( i ) + {\sum }_{i} {g}^{b} ( i ) + γ} \qquad ( 3 )$$

where g^{b}(i) is the intrapulmonary fissure category true value corresponding to the i-th element (that is, the intrapulmonary fissure category grout truth); p^{b}(i) is the intrapulmonary fissure category to which the i-th element belongs; the value of b may be 1 (i.e., the category value of intrapulmonary fissure) or 0 (i.e., the category value of non-pulmonary fissure); and γ is 1e−5. What needs to be noted here is that, because the classification of intrapulmonary fissure belongs to a dichotomy, b only needs to be calculated as one of 1 and 0.

Further, in order to facilitate the subsequent neural network to learn the features of the lung lobes and speed up the convergence, the difference between the lung region and the extra-pulmonary region in the sample image can be enhanced before the sample image is input to the first neural network model. In implementations, the above method 200 further includes:

206. Inputting the sample image to a second neural network model to obtain a sample lung segmentation result.

The sample lung segmentation result includes the identified lung region and extra-pulmonary region.

208. Altering the gray value of the first region corresponding to the extra-pulmonary region in the sample image to enhance the difference between the first region and the second region corresponding to the lung region in the sample image.

For the processing procedure on the sample image by the second neural network model, reference may be made to the processing procedure on the image to be processed containing the lung image by the second neural network model in the above various example embodiments, and details are not repeated herein. For the training process of the second neural network model, reference may be made to corresponding content in the above various example embodiments, and details are not repeated herein.

In the above 208, for the step of altering the gray value of the first region corresponding to the extra-pulmonary region in the sample image, reference may be made to the step of altering the gray value of the first region corresponding to the extra-pulmonary region in the image to be processed in the above various example embodiments, and details are not repeated herein.

Further, the first neural network model further includes an input layer and an output layer. The first network layer is located between the input layer and the output layer.

The positions and the number of the first network layers in the first neural network model can be set according to actual needs, and are not limited in the example embodiments of the present disclosure.

Through research, the inventor found that if the first network layer is provided at a position close to the front in the first neural network model, as the feature extraction (such as convolution) is continuously performed subsequently, the coordinate information will be weakened, resulting in reduced improvement in the accuracy of lung lobe segmentation. If the first network layer is provided prior to the output layer and connected to the output layer, the accuracy of lung lobe segmentation can be greatly improved.

In an example embodiment, only one first network layer is provided in the first neural network model, and the first network layer is connected to the output layer.

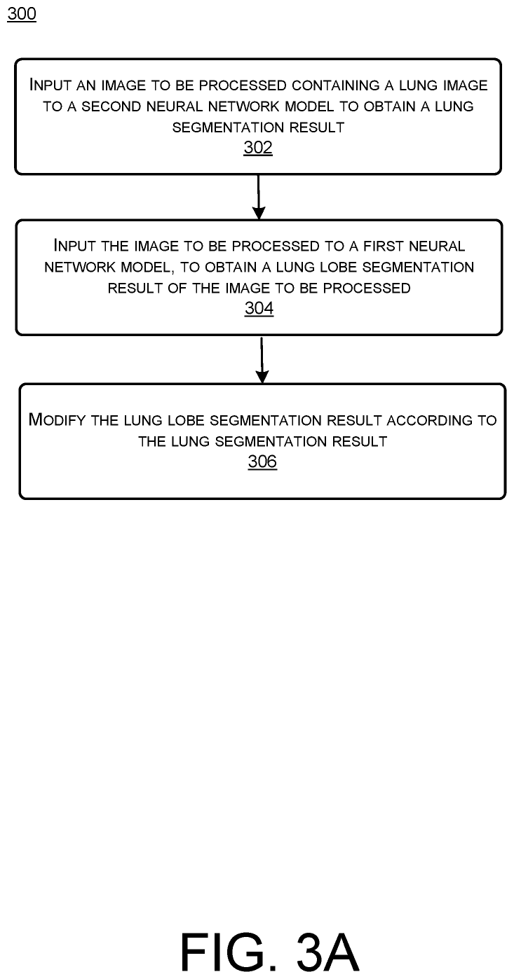

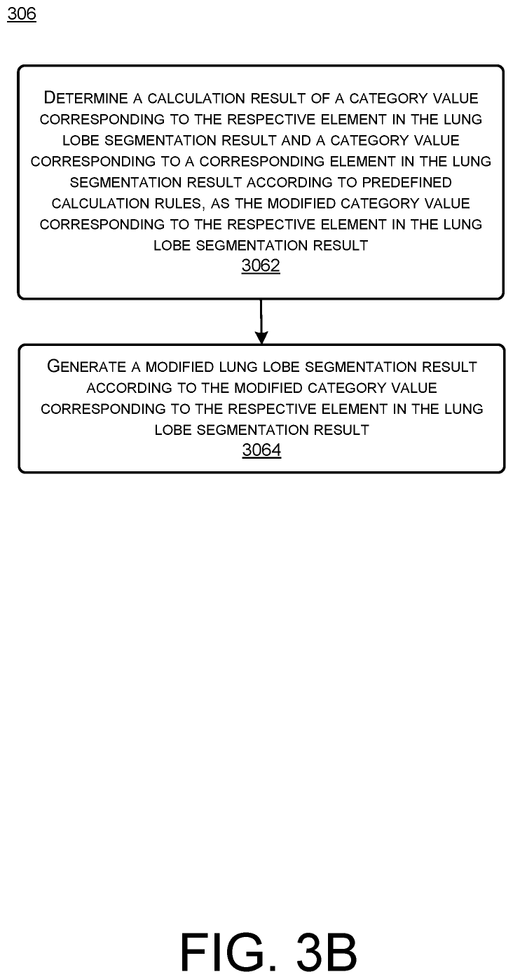

FIG. 3A illustrates a schematic flowchart of a lung lobe segmentation method 300 provided by another example embodiment of the present disclosure. As shown in FIG. 3, the method 300 includes:

302. Inputting an image to be processed containing a lung image to a second neural network model to obtain a lung segmentation result.

The lung segmentation result includes the identified lung region.

304. Inputting the image to be processed to a first neural network model, to obtain a lung lobe segmentation result of the image to be processed.

306. Modifying the lung lobe segmentation result according to the lung segmentation result.

In an implementable solution, in the above 304, the first neural network model may be a neural network model based on v-net as basic network architecture.

In order to improve the accuracy of lung lobe segmentation, the first neural network model may be the first neural network model mentioned in the above various example embodiments.

For implementations of the above steps 302, 304, and 306, reference may be made to corresponding content in the above various example embodiments, and details are not repeated herein.

In the technical solutions provided by the example embodiments of the present disclosure, the situation of extra-pulmonary misclassification can be reduced through the assistance of the second neural network model.

Further, the lung segmentation result further includes an identified extra-pulmonary region. The category value corresponding to the respective element in the lung region in the lung segmentation result is assigned the first value. The category value corresponding to the respective element in the extra-pulmonary region is assigned the second value.

Referring to FIG. 3B, in implementations, the “modifying the lung lobe segmentation result according to the lung segmentation result” in the above 306 may be implemented by the following steps:

3062. Determining a calculation result of a category value corresponding to the respective element in the lung lobe segmentation result and a category value corresponding to a corresponding element in the lung segmentation result according to predefined calculation rules, as the modified category value corresponding to the respective element in the lung lobe segmentation result.

3064. Generating a modified lung lobe segmentation result according to the modified category value corresponding to the respective element in the lung lobe segmentation result.

The calculation rules include: the calculation result of the first value and a third value is the third value; and the calculation result of the second value and a fourth value is the second value.

What needs to be noted here is that, for the content that is not described in detail in the steps in the methods provided in the example embodiments of the present disclosure, reference may be made to the corresponding content in the above various example embodiments, and details are not be repeated here. In addition, the methods provided in the example embodiments of the present disclosure may include other or all steps in the above various example embodiments in addition to the above steps. Reference may be made to the corresponding content of the above various example embodiments, and details are not repeated herein.

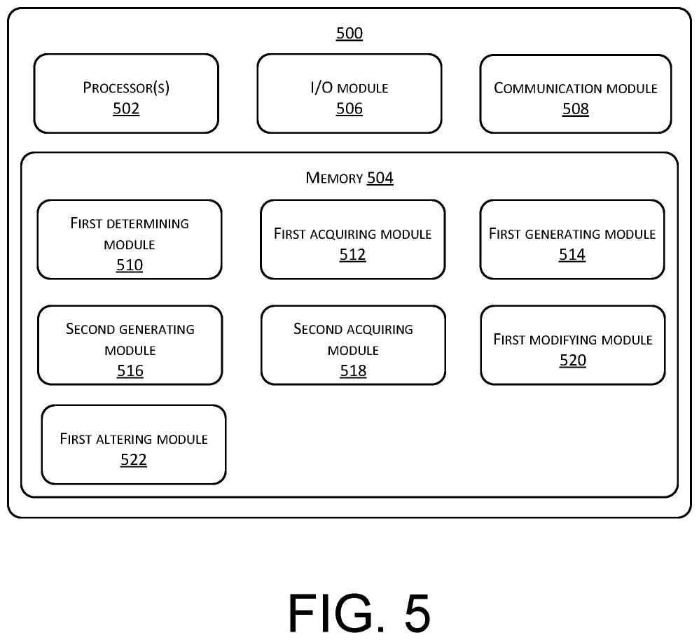

FIG. 5 shows a structural block diagram of a lung lobe segmentation apparatus 500 provided by an example embodiment of the present disclosure. As shown in FIG. 5, the apparatus 500 may include one or more processors 502, a memory 504, an input/output module 506, and a communication module 508. The input/output module 506 is configured to receive data/signal to be processed and to output the processed data/signal. The communication module 508 is configured to allow the apparatus 500 to communicate with other devices (not shown) over a network (not shown). The memory 504 stores thereon computer-executable modules executable by the one or more processors 502. The computer-executable modules may include a first determining module 510 and a first acquiring module 512, where

The first determining module 510 is configured to determine a first neural network model;

The first acquiring module 512 is configured to input an image to be processed containing a lung image to the first neural network model to obtain a lung lobe segmentation result of the image to be processed.

The first network layer in the first neural network model is configured to add an additional channel with coordinate information to the input data input to the first network layer, and to determine the output data of the first network layer based on the input data after the additional channel is added thereto.

In the technical solutions provided by the example embodiments of the present disclosure, the first network layer is designed in the first neural network model for lung lobe segmentation. The first network layer adds the additional channel with coordinate information to the input data input to the first network layer, and determines the output data of the first network layer based on the input data after the additional channel is added thereto. The relative position distribution of the five lung lobes in the lung image is relatively fixed. The introduction of coordinate information may enable the neural network model to learn the coordinate features and global information, which play the role of coordinate guidance, effectively reducing the misclassification within the lung, and improving the accuracy of lung lobe segmentation.

Further, the input data is a multi-channel feature map; and the input data includes a first channel map.

The above apparatus 500 further includes:

A first generating module 514 is configured to generate coordinate information of the respective element according to the position of the respective element in the first channel map;

A second generating module 516 is configured to generate the additional channel according to the position of the respective element in the first channel map and coordinate information of the respective element.

Further, the image to be processed is a three-dimensional image. The first channel map is a three-dimensional channel map. The coordinate information includes a first coordinate on a first axis, a second coordinate on a second axis, and a third coordinate on a third axis, where the first axis, the second axis, and the third axis intersect one with another.

The second generating module 516 is configured to:

generate a first additional channel corresponding to the first axis according to the position of the respective element in the first channel map and the first coordinate of the respective element;

generate a second additional channel corresponding to the second axis according to the position of the respective element in the first channel map and the second coordinate of the respective element;

generate a third additional channel corresponding to the third axis according to the position of the respective element in the first channel map and a third coordinate of the respective element.

Further, the first generating module 514 is configured to:

perform normalization processing on the position information of the respective element in the first channel map to obtain the coordinate information of the respective element.

Further, the above apparatus 500 further includes:

A second acquiring module 518 is configured to input the image to be processed to a second neural network model to obtain a lung segmentation result, where the lung segmentation result includes an identified lung region and/or extra-pulmonary region;

A first modifying module 520 is configured to modify the lung lobe segmentation result according to the lung segmentation result.

Further, the first modifying module 520 is configured to:

determine an extra-pulmonary misclassified region in the lung lobe segmentation result according to the lung region when the lung segmentation result includes the identified lung region;

determine the extra-pulmonary misclassified region in the lung lobe segmentation result according to the extra-pulmonary region when the lung segmentation result includes the identified extra-pulmonary region;

modify the category of the extra-pulmonary misclassified region in the lung lobe segmentation result to the extra-pulmonary region category to obtain a modified lung lobe segmentation result.

Further, the lung segmentation result includes the identified lung region and extra-pulmonary region. The category value corresponding to the respective element in the lung region in the lung segmentation result is assigned the first value. The category value corresponding to the respective element in the extra-pulmonary region is assigned the second value.

The first modifying module 520 is configured to:

determine a calculation result of a category value corresponding to the respective element in the lung lobe segmentation result and a category value corresponding to a corresponding element in the lung segmentation result according to predefined calculation rules, as the modified category value corresponding the respective element of the lung lobe segmentation result;

generate a modified lung lobe segmentation result according to the modified category value corresponding to the respective element in the lung lobe segmentation result.

The calculation rules include: the calculation result of the first value and a third value is the third value; and the calculation result of the second value and a fourth value is the second value.

Further, the lung segmentation result includes the identified lung region and extra-pulmonary region. The above apparatus 500 further includes a first altering module 522 configured to:

alter the gray value of a first region corresponding to the extra-pulmonary region in the image to be processed to enhance the difference between the first region and the second region corresponding to the lung region in the image to be processed, before the image to be processed is input to the first neural network model.

Further, the first altering module 522 is configured to:

uniformly set the gray value of the first region to a set value greater than a first preset threshold in the image to be processed.

Further, the first neural network model further includes an input layer and an output layer. The first network layer is located between the input layer and the output layer, and is connected to the output layer.

Further, the first network layer is configured to perform feature extraction on the input data after the additional channel is added thereto to obtain a third feature map as the output data of the first network layer.

What needs to be noted here is that the lung lobe segmentation apparatus provided by the above example embodiments can implement the technical solutions described in the above method example embodiments. For the implementation principles of the above modules or units, reference may be made to the corresponding content in the above method example embodiments, and details will not be repeated herein.

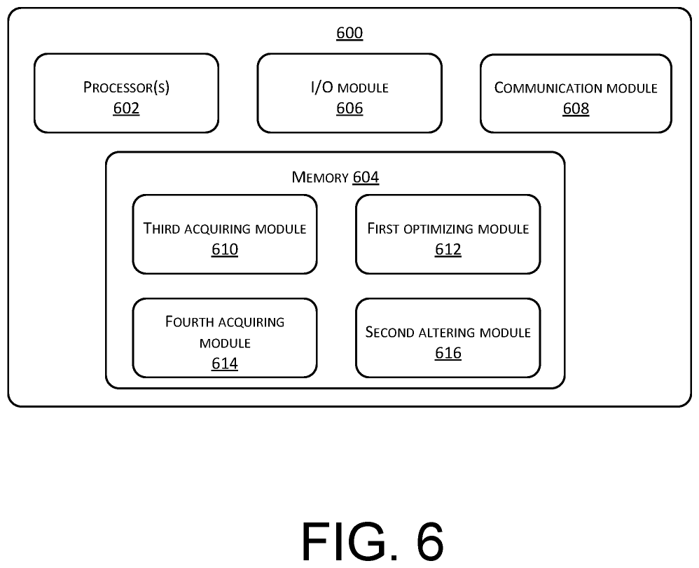

FIG. 6 illustrates a structural block diagram of a model training apparatus provided by another example embodiment of the present disclosure. As shown in FIG. 6, the apparatus 600 may include one or more processors 602, a memory 604, an input/output module 606, and a communication module 608. The input/output module 606 is configured to receive data/signal to be processed and to output the processed data/signal. The communication module 608 is configured to allow the apparatus 600 to communicate with other devices (not shown) over a network (not shown). The memory 604 stores thereon computer-executable modules executable by the one or more processors 602. The computer-executable modules may include a third acquiring module 610 and a first optimizing module 612, where

The third acquiring module 610 is configured to input a sample image containing a lung image to a first neural network model, to obtain a lung lobe segmentation prediction result of the sample image;

The first optimizing module 612 is configured to perform parameter optimization on the first neural network model according to the lung lobe segmentation prediction result and the lung segmentation ground truth result of the sample image.

The first neural network model is configured for lung lobe segmentation. The first network layer in the first neural network model is configured to add the additional channel with coordinate information to the input data input to the first network layer, and to determine the output data of the first network layer based on the input data to which the additional channel is added.

In the technical solutions provided in the example embodiments of the present disclosure, the first network layer is designed in the first neural network model for lung lobe segmentation. The first network layer in the first neural network model is configured to add the additional channel with coordinate information to the input data input to the first network layer, and to determine the output data of the first network layer based on the input data to which the additional channel is added. The relative position distribution of the five lung lobes in the lung image is relatively fixed. The introduction of coordinate information may enable the neural network model to learn the coordinate features and global information, which play the role of coordinate guidance, effectively reducing the misclassification within the lung, and improving the accuracy of lung lobe segmentation.

Further, the lung lobe segmentation prediction result includes the lung lobe classification prediction result and the intrapulmonary fissure classification prediction result. The lung lobe segmentation ground truth result includes the lung lobe classification ground truth result and the intrapulmonary fissure classification ground truth result.

The first optimizing module 612 is configured to:

calculate a first loss value according to the lung lobe classification prediction result and the lung lobe classification ground truth result;

calculate a second loss value according to the intrapulmonary fissure classification prediction result and the intrapulmonary fissure classification ground truth result;

perform parameter optimization on the first neural network model based on an integration of the first loss value and the second loss value.

Further, the first optimizing module 612 is configured to:

execute the first Dice coefficient loss function to obtain a first loss value, with the lung lobe classification prediction result and the lung lobe classification ground truth result as the input of the first Dice coefficient loss function;

execute the second Dice coefficient loss function to obtain a second loss value, with the intrapulmonary fissure classification prediction result and the intrapulmonary fissure classification ground truth result as the input of the second Dice coefficient loss function.

Further, the above apparatus 600 further includes a fourth acquiring module 614 and a second altering module 616, where

The fourth acquiring module 614 is configured to, before the sample image is input to the first neural network model, input the sample image to a second neural network model to obtain the lung segmentation result, where the lung segmentation result includes the identified lung region and extra-pulmonary region;

The second altering module 616 is configured to, before the sample image is input to the first neural network model, alter the gray value of the first region corresponding to the extra-pulmonary region in the sample image, to enhance the difference between the first region and the second region corresponding to the lung region in the sample image.

Further, the first neural network model further includes an input layer and an output layer.

The first network layer is located between the input layer and the output layer, and is connected to the output layer.

What needs to be noted here is that the model training apparatus provided by the above example embodiments can implement the technical solutions described in the above method example embodiments. For the implementation principles of the above modules or units, reference may be made to the corresponding content in the above method example embodiments, and details are not repeated herein.

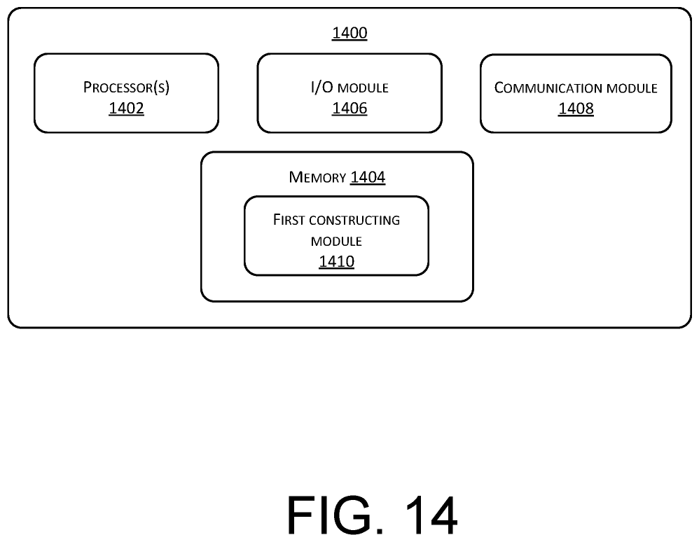

As shown in FIG. 14, another example embodiment of the present disclosure provides a model construction apparatus 1400. The apparatus 1400 may include one or more processors 1402, a memory 1404, an input/output module 1406, and a communication module 1408. The input/output module 1406 is configured to receive data/signal to be processed and to output the processed data/signal. The communication module 1408 is configured to allow the apparatus 1400 to communicate with other devices (not shown) over a network (not shown). The memory 1404 stores thereon computer-executable modules executable by the one or more processors 1402. The computer-executable modules may include:

A first constructing module 1410 is configured to construct at least one network layer to obtain a first neural network model for lung lobe segmentation.

The at least one network layer includes at least one first network layer. The first network layer is configured to add an additional channel with coordinate information to input data input to the first network layer, and to determine the output data of the first network layer based on the input data after the additional channel is added thereto.

In the technical solutions provided by the example embodiments of the present disclosure, the first network layer is designed in the first neural network model for lung lobe segmentation. The first network layer adds the additional channel having coordinate information to the input data input to the first network layer, and determines the output data of the first network layer based on input data after the additional channel is added thereto. The relative position distribution of the five lung lobes in the lung image is relatively fixed. The introduction of coordinate information may enable the neural network model to learn the coordinate features and global information, which play the role of coordinate guidance, effectively reducing the misclassification within the lung, and improving the accuracy of lung lobe segmentation.

Further, the number of the first network layers in the at least one network layer is one. The at least one network layer includes an input layer and an output layer. The first network layer is located between the input layer and the output layer, and the first network layer is connected to the output layer.

What needs to be noted here is that the model constructing apparatus provided by the above example embodiments can implement the technical solutions described in the above method example embodiments. For the implementation principles of the above modules or units, reference may be made to the corresponding content in the above method example embodiments, and are not repeated herein.

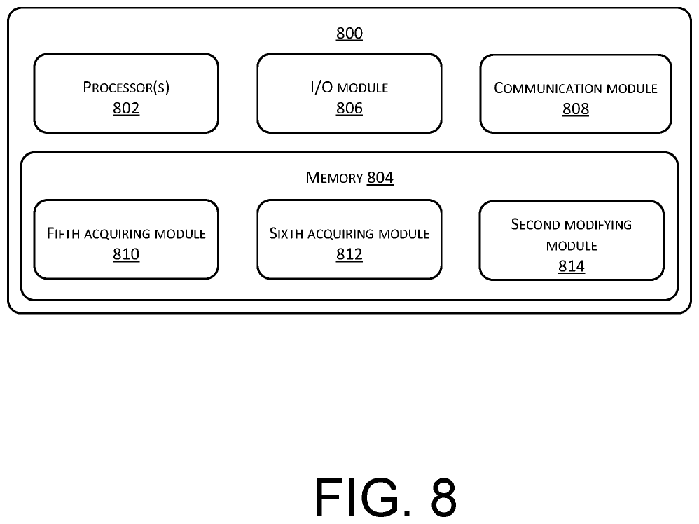

FIG. 8 illustrates a structural block diagram of a lung lobe segmentation apparatus provided by another example embodiment of the present disclosure. As shown in FIG. 8, the apparatus 800 may include one or more processors 802, a memory 804, an input/output module 806, and a communication module 808. The input/output module 806 is configured to receive data/signal to be processed and to output the processed data/signal. The communication module 808 is configured to allow the apparatus 800 to communicate with other devices (not shown) over a network (not shown). The memory 804 stores thereon computer-executable modules executable by the one or more processors 802. The computer-executable modules may include a fifth acquiring module 810, a sixth acquiring module 812, and a second modifying module 814, where

The fifth acquiring module 810 is configured to input an image to be processed containing a lung image to a second neural network model to obtain a lung segmentation result, where the lung segmentation result includes the identified lung region;

The sixth acquiring module 812 is configured to input the image to be processed to a first neural network model, to obtain a lung lobe segmentation result of the image to be processed;

The second modifying module 814 is configured to modify the lung lobe segmentation result according to the lung segmentation result.

In the example embodiment, with the assistance of the second neural network model, the situation of extra-pulmonary misclassification can be reduced.

Further, the lung segmentation result further includes an identified extra-pulmonary region. The category value corresponding to the respective element in the lung region in the lung segmentation result is assigned the first value, and the category value corresponding to the respective element in the extra-pulmonary region is assigned the second value;

The second modifying module 814 is configured to:

determine a calculation result of a category value corresponding to the respective element in the lung lobe segmentation result and a category value corresponding to a corresponding element in the lung segmentation result according to predefined calculation rules, as the modified category value corresponding to the respective element in the lung lobe segmentation result;

generate a modified lung lobe segmentation result according to the modified category value corresponding to the respective element in the lung lobe segmentation result;

The calculation rules include: the calculation result of the first value and a third value is the third value; and the calculation result of the second value and a fourth value is the second value.

What needs to be noted here is that the lung lobe segmentation apparatus provided by the above example embodiments can implement the technical solutions described in the above method example embodiments. For the implementation principles of the above modules or units, reference may be made to the corresponding content in the above method example embodiments, and details are not repeated herein.

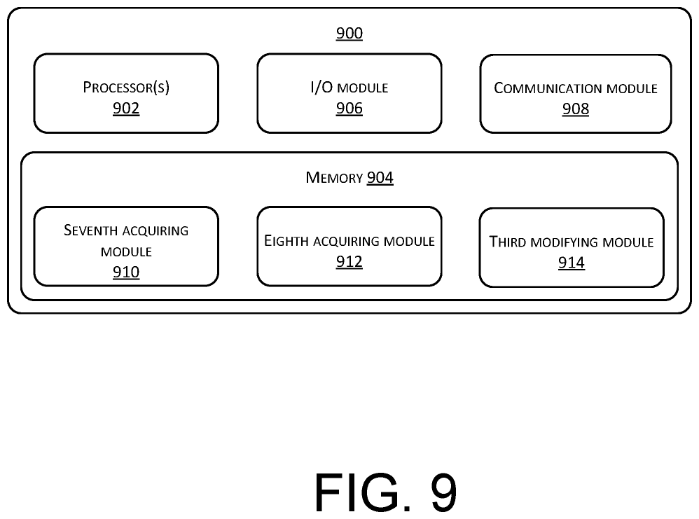

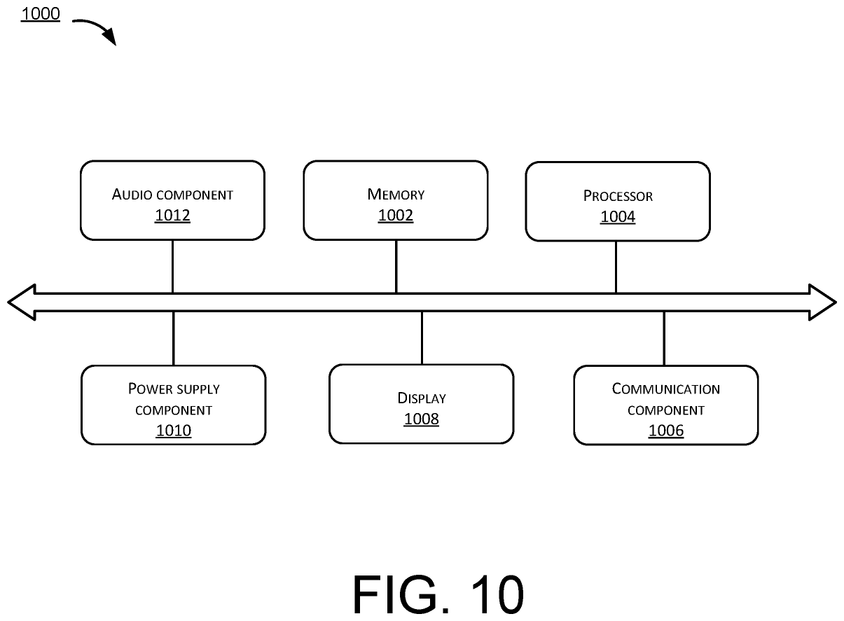

FIG. 9 illustrates a structural block diagram of a segmentation apparatus 900 provided by another example embodiment of the present disclosure. As shown in FIG. 9, the apparatus 900 may include one or more processors 902, a memory 904, an input/output module 906, and a communication module 908. The input/output module 906 is configured to receive data/signal to be processed and to output the processed data/signal. The communication module 908 is configured to allow the apparatus 900 to communicate with other devices (not shown) over a network (not shown). The memory 904 stores thereon computer-executable modules executable by the one or more processors 902. The computer-executable modules may include:

A seventh acquiring module 910 is configured to input an image to be processed containing a target object image to a second neural network model to obtain a target segmentation result, where the target segmentation result includes an identified target object region;

An eighth acquiring module 912 is configured to input the image to be processed to a first neural network model, and obtain an identified unit region corresponding to the unit constituting the target object;

A third modifying module 914 is configured to modifying the unit region according to the target segmentation result.

In the example embodiment, with the assistance of the second neural network model, the situation of misclassification other than the target object can be reduced.

Further, the target segmentation result further includes an identified region other than the target object. The category value corresponding to the respective element in the target object region in the target segmentation result is assigned the first value. The category value corresponding to the respective element of the region other than the target object is assigned the second value.

The third modifying module 914 is configured to:

determine a calculation result of a calculation result of category value corresponding to the respective element in the unit region and a category value corresponding to a category value corresponding to the respective element in the target segmentation result according to predefined calculation rules, as the modified category value corresponding of the respective element in the unit region;

generate a modified unit region according to the modified category value corresponding to the respective element in the unit region.

The calculation rules include: the calculation result of the first value and a third value is the third value; and the calculation result of the second value and a fourth value is the second value.

What needs to be noted here is that the segmentation apparatus provided in the above example embodiments can implement the technical solutions described in the above method example embodiments. For the implementation principles of the above modules or units, reference may be made to corresponding content in the above method example embodiments, and details are not repeated herein.

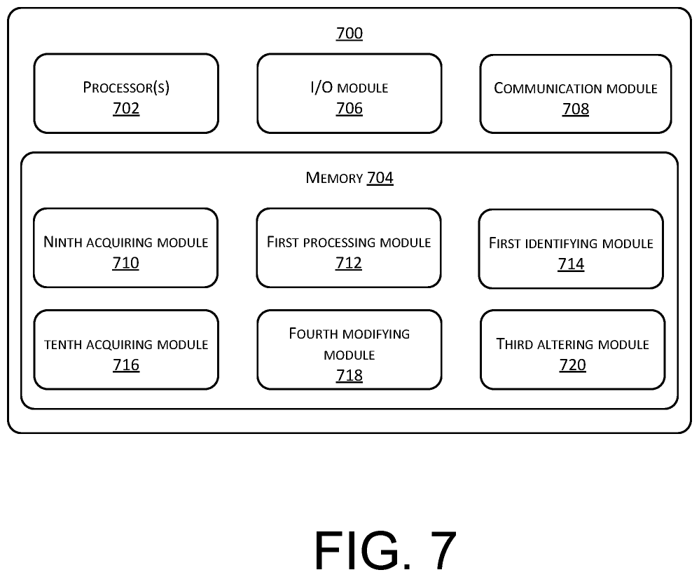

FIG. 7 is a structural block diagram of a segmentation apparatus 700 provided by an example embodiment of the present disclosure. As shown in the figure, the apparatus 700 may include one or more processors 702, a memory 704, an input/output module 706, and a communication module 708. The input/output module 706 is configured to receive data/signal to be processed and to output the processed data/signal. The communication module 708 is configured to allow the apparatus 700 to communicate with other devices (not shown) over a network (not shown). The memory 704 stores thereon computer-executable modules executable by the one or more processors 702. The computer-executable modules may include a ninth acquiring module 710, a first processing module 712, and a first identifying module 714, where

The ninth acquiring module 710 is configured to acquire an image to be processed containing a target object image;

The first processing module 712 is configured to process the image to be processed, where the processing procedure includes an operation of adding an additional channel with coordinate information;

The first identifying module 714 is configured to identify a unit region corresponding to a unit constituting the target object based on a processing result of the image to be processed.

Further, the first processing module 712 is configured to:

perform feature extraction on the image to be processed to obtain a first feature map;

add the additional channel to the first feature map to obtain a second feature map.

Further, the first processing module 712 is further configured to:

perform feature extraction on the second feature map to obtain a third feature map.

Further, the step of adding the additional channel is implemented by a first network layer in the first neural network model.

Further, the first neural network model includes an input layer and an output layer. The first network layer is located between the input layer and the output layer and is connected to the output layer.

Further, the apparatus 700 further includes:

A tenth acquiring module 716 is configured to input the image to be processed to a second neural network model to obtain a target segmentation result, where the target segmentation result includes the identified target object region;

A fourth modifying module 718 is configured to modify the unit region according to the target segmentation result.

Further, the above apparatus 700 further includes:

A third altering module 720 is configured to alter the gray value of a first region corresponding to a region other than the target object in the image to be processed to enhance the difference between the first region and the second region corresponding to the target object region in the image to be processed.

FIG. 10 illustrates a schematic structural diagram of an electronic device 1000 provided by an example embodiment of the present disclosure. As shown in the figure, the electronic device 1000 includes a memory 1002 and a processor 1004. The memory 1002 may be configured to store various other data to support operations on the electronic device 1000. Examples of such data include instructions of any application or method for operating on the electronic device 1000. The memory 1002 may be implemented by any type of volatile or non-volatile storage device or a combination thereof, such as Static Random Access Memory (SRAM), Electrically Erasable Programmable Read-only Memory (EEPROM), Erasable Programmable Read-only Memory (EPROM), Programmable Read-only Memory (PROM), Read-only Memory (ROM), magnetic memory, flash memory, magnetic disk or optical disk.

The processor 1004 is coupled to the memory 1002, and is configured to execute programs stored in the memory 1002, so as to:

determine a first neural network model;

input an image to be processed containing a lung image to the first neural network model to obtain a lung lobe segmentation result of the image to be processed;

wherein the first network layer in the first neural network model is configured to add an additional channel with coordinate information to the input data input to the first network layer, and to determine output data of the first network layer based on the input data after the additional channel is added thereto.

When the processor 1004 executes programs in the memory 1002, the processor 1004 can implement other functions in addition to the above functions. For details, reference may be made to the descriptions of the above various example embodiments.

Further, as shown in FIG. 10, the electronic device 1000 further includes a communication component 1006, a display 1008, a power supply component 1010, an audio component 1012, and other components. Merely some components are shown schematically in FIG. 10, which does not mean that the electronic device 1000 includes merely the components shown in FIG. 10.

Accordingly, an example embodiment of the present disclosure further provides a computer-readable storage medium storing computer programs that can implement the steps or functions of the lung lobe segmentation method provided by the above various example embodiments when the computer programs are executed by a computer.

Referring to FIG. 10, the processor 1004 is coupled to the memory 1002, and is configured to execute programs stored in the memory 1002, so as to:

input a sample image containing a lung image to a first neural network model, to obtain a lung lobe segmentation prediction result of the sample image;

perform parameter optimization on the first neural network model according to the lung lobe segmentation prediction result and the lung lobe segmentation ground truth result of the sample image.

The first neural network model is configured for lung lobe segmentation. The first network layer in the first neural network model is configured to add the additional channel with coordinate information to the input data input to the first network layer, and to determine the output data of the first network layer based on the input data to which the additional channel is added.

When the processor 1004 executes programs in the memory 1002, the processor 1004 can implement other functions in addition to the above functions. For details, reference may be made to the descriptions of the above various example embodiments.

Accordingly, an example embodiment of the present disclosure further provides a computer-readable storage medium storing computer programs that can implement the steps or functions of the model training method provided by the above various example embodiments when the computer programs are executed by a computer.

FIG. 10 illustrates, the processor 1004 is coupled to the memory 1002, and is configured to execute programs stored in the memory 1002, so as to:

construct at least one network layer to obtain a first neural network model for lung lobe segmentation.

The at least one network layer includes at least one first network layer. The first network layer is configured to add an additional channel with coordinate information to the input data input to the first network layer, and to determine the output data of the first network layer based on the input data after the additional channel is added thereto.

When the processor 1004 executes programs in the memory 1002, the processor 1004 can implement other functions in addition to the above functions. For details, reference may be made to the descriptions of the above various example embodiments.

Accordingly, an example embodiment of the present disclosure further provides a computer-readable storage medium storing computer programs that can implement the steps or functions of the model construction method provided by the above various example embodiments when the computer programs are executed by a computer.

Referring to FIG. 10, the processor 1004 is coupled to the memory 1002, and is configured to execute programs stored in the memory 1002, so as to:

input an image to be processed containing a lung image to a second neural network model to obtain a lung segmentation result, where the lung segmentation result includes the identified lung region;

input the image to be processed to a first neural network model to obtain a lung lobe segmentation result of the image to be processed;

modify the lung lobe segmentation result according to the lung segmentation result.

When the processor 1004 executes programs in the memory 1002, the processor 1004 can implement other functions in addition to the above functions. For details, reference may be made to the descriptions of the above various example embodiments.

Accordingly, an example embodiment of the present disclosure further provides a computer-readable storage medium storing computer programs that can implement the steps or functions of the lung lobe segmentation method provided by the above various example embodiments when the computer programs are executed by a computer.

Referring to FIG. 10, the processor 1004 is coupled to the memory 1002, and is configured to execute programs stored in the memory 1002, so as to:

input an image to be processed containing a target object image to a second neural network model to obtain a target segmentation result, where the target segmentation result includes the identified target object region;

input the image to be processed to a first neural network model to obtain an identified unit region corresponding to the unit constituting the target object;

modify the unit region according to the target segmentation result.

When the processor 1004 executes programs in the memory 1002, the processor 1004 can implement other functions in addition to the above functions. For details, reference may be made to the descriptions of the above various example embodiments.

Accordingly, an example embodiment of the present disclosure further provides a computer-readable storage medium storing computer programs that can implement the steps or functions of the segmentation method provided by the above various example embodiments when the computer programs are executed by a computer.

Referring to FIG. 10, the processor 1004 is coupled to the memory 1002, and is configured to execute programs stored in the memory 1002, so as to:

acquire an image to be processed containing a target object image;

process the image to be processed, where the processing procedure includes an operation of adding an additional channel with coordinate information;

identify a unit region corresponding to a unit constituting the target object, based on a processing result of the image to be processed.

When the processor 1004 executes programs in the memory 1002, the processor 1004 can implement other functions in addition to the above functions. For details, reference may be made to the descriptions of the above various example embodiments.

Accordingly, an example embodiment of the present disclosure further provides a computer-readable storage medium storing computer programs that can implement the steps or functions of the segmentation method provided by the above various example embodiments when the computer programs are executed by a computer.

The device example embodiments described above are only schematic. The units described as separate components may or may not be physically separated. The components shown as units may or may not be physical units, that is, the components may be located in one place or distributed across multiple network elements. Some or all of the modules may be selected according to actual needs to achieve the objective of the solutions of the example embodiments. Those of ordinary skill in the art can understand and implement without creative efforts.

Through the description of the above implementation manners, those of ordinary skill in the art can clearly understand that the implementation manners may be implemented by means of software plus a necessary universal hardware platform, and of course, also by means of hardware. Based on such an understanding, the above technical solution, or the portion that contributes to the conventional techniques, may be embodied in the form of a software product. The software product can be stored in a computer-readable storage medium, such as ROM/RAM, magnetic disc, optical disc, and the like, including instructions for causing a computer device (which may be a personal computer, a server, or a network device, etc.) to execute the methods described in various example embodiments or certain parts of the example embodiments. As defined herein, the computer readable media does not include transitory media, such as modulated data signals and carrier waves.

Finally, what needs to be noted is that the above example embodiments are only used to describe the technical solutions of the present disclosure but are not limitations thereto. Although the present disclosure is described in detail with reference to the above example embodiments, those of ordinary skill in the art should understand that he technical solutions described in the above various example embodiments may still be modified, or some of the technical features therein may be equivalently replaced. Such modifications or replacements do not deviate the corresponding technical solutions from the scope of the technical solutions of the various example embodiments of the present disclosure.

### EXAMPLE CLAUSES

Clause 1. A lung lobe segmentation method, comprising: determining a first neural network model; and inputting an image to be processed containing a lung image to the first neural network model to obtain a lung lobe segmentation result of the image to be processed; wherein the first network layer in the first neural network model is configured to add an additional channel with coordinate information to input data input to the first network layer, and to determine output data of the first network layer based on the input data after the additional channel is added thereto.

Clause 2. The method according to clause 1, wherein the input data is a multi-channel feature map; and the multi-channel feature map includes a first channel map; the above method further comprising: generating coordinate information of respective element according to a position of the respective element in the first channel map; and generating the additional channel according to the position of the respective element in the first channel map and the coordinate information of the respective element.

Clause 3. The method according to clause 2, wherein the image to be processed is a three-dimensional image; the first channel map is a three-dimensional channel map; and the coordinate information includes a first coordinates on a first axis, a second coordinate on a second axis, and a third coordinate on a third axis; and the first axis, the second axis, and the third axis intersect one with another; and generating the additional channel according to the position of the respective element in the first channel map and the coordinate information of the respective element includes: generating a first additional channel corresponding to the first axis according to the position of the respective element in the first channel map and the first coordinate of respective element; generating a second additional channel corresponding to the second axis according to the position of the respective element in the first channel map and the second coordinate of the respective element; and generating a third additional channel corresponding to the third axis according to the position of the respective element in the first channel map and the third coordinate of the respective element.

Clause 4. The method according to clause 2, wherein generating the coordinate information of the respective element according to the position of the respective element in the first channel map includes: performing normalization processing on position information of the respective element in the first channel map to obtain the coordinate information of the respective element.

Clause 5. The method according to any one of clauses 1 to 4, further comprising: inputting the image to be processed to a second neural network model to obtain a lung segmentation result, wherein the lung segmentation result includes an identified lung region and/or extra-pulmonary region; and modifying the lung lobe segmentation result according to the lung segmentation result.

Clause 6. The method according to clause 5, wherein modifying the lung lobe segmentation result according to the lung segmentation result includes: when the lung segmentation result includes the identified lung region, determining an extra-pulmonary misclassified region in the lung lobe segmentation result according to the lung region; when the lung segmentation result includes the identified extra-pulmonary region, determining the extra-pulmonary misclassified region in the lung lobe segmentation result according to the extra-pulmonary region; and in the lung lobe segmentation result, modifying a category of the extra-pulmonary misclassified region to an extra-pulmonary region category to obtain a modified lung lobe segmentation result.

Clause 7. The method according to clause 5, wherein the lung segmentation result includes the identified lung region and extra-pulmonary region; a category value corresponding to respective element in the lung region in the lung segmentation result is assigned a first value; and a category value corresponding to respective element in the extra-pulmonary region is assigned a second value; and modifying the lung lobe segmentation result according to the lung segmentation result includes: determining a calculation result of the category value corresponding to the respective element in the lung segmentation result and a category value corresponding to corresponding element in the lung segmentation result according to predefined calculation rules, as a modified category value corresponding to the respective element in the lung lobe segmentation result; and generating a modified lung lobe segmentation result according to the modified category value corresponding to the respective element in the lung lobe segmentation result; wherein the calculation rules include: a calculation result of the first value and a third value is the third value; and a calculation result of the second value and a fourth value is the second value.

Clause 8. The method according to clause 5, wherein the lung lobe segmentation result includes the identified lung region and extra-pulmonary region; and prior to inputting the image to be processed to the first neural network model, the method further includes: altering a gray value of a first region corresponding to the extra-pulmonary region in the image to be processed to enhance a difference between the first region and a second region corresponding to the lung region in the image to be processed.

Clause 9. The method according to clause 8, wherein altering the gray value of the first region corresponding to the extra-pulmonary region in the image to be processed to enhance the difference between the first region and the second region corresponding to the lung region in the image to be processed includes: uniformly setting the gray value of the first region in the image to be processed to a set value greater than a first preset threshold.

Clause 10. The method according to any one of clauses 1 to 4, wherein the first neural network model further includes an input layer and an output layer; and the first network layer is located between the input layer and the output layer, and is connected to the output layer.

Clause 11. The method according to any one of clauses 1 to 4, wherein the step of determining the output data of the first network layer based on the input data after the additional channel is added thereto includes: performing feature extraction on the input data after the additional channel is added thereto to obtain a third feature map as the output data of the first network layer.

Clause 12. A model training method, comprising: inputting a sample image containing a lung image to a first neural network model to obtain a lung lobe segmentation prediction result of the sample image; and performing parameter optimization on the first neural network model according to the lung lobe segmentation prediction result and a lung lobe segmentation ground truth result of the sample image; wherein the first neural network model is configured for lung lobe segmentation; a first network layer in the first neural network model is configured to add an additional channel with coordinate information to input data input to the first network layer, and to determine output data of the first network layer based on the input data to which the additional channel is added.

Clause 13. The method according to clause 12, wherein the lung lobe segmentation prediction result includes a lung lobe classification prediction result and an intrapulmonary fissure classification prediction result; and the lung lobe segmentation ground truth result includes a lung lobe classification ground truth result and an intrapulmonary fissure classification ground truth result; and performing parameter optimization on the first neural network model according to the lung lobe segmentation prediction result and the lung lobe segmentation ground truth result of the sample image includes: calculating a first loss value according to the lung lobe classification prediction result and the lung lobe classification ground truth result; calculating a second loss value according to the intrapulmonary fissure classification prediction result and the intrapulmonary fissure classification ground truth result; and performing parameter optimization on the first neural network model integrate the first loss value and the second loss value based on an integration of the first loss value and the second loss value.

Clause 14. The method according to clause 13, wherein calculating the first loss value according to the lung lobe classification prediction result and the lung lobe classification ground truth result includes: executing a first Dice coefficient loss function to obtain the first loss value, with the lung lobe classification prediction result and the lung lobe classification ground truth result as inputs of the first Dice coefficient loss function; and wherein calculating the second loss value according to the intrapulmonary fissure classification prediction result and the intrapulmonary fissure classification ground truth result includes: executing a second Dice coefficient loss function to obtain the second loss value, with the intrapulmonary fissure classification prediction result and the intrapulmonary fissure classification ground truth result as inputs of the second Dice coefficient loss function.

Clause 15. The method according to any one of clauses 12 to 14, prior to inputting the sample image to the first neural network model, the method further comprising: inputting the sample image to a second neural network model to obtain a lung segmentation result, where the lung segmentation result includes an identified lung region and extra-pulmonary region; and altering a gray value of a first region corresponding to the extra-pulmonary region in the sample image to enhance a difference between the first region and a second region corresponding to the lung region in the sample image.

Clause 16. The method according to any one of clauses 12 to 14, wherein the first neural network model further includes an input layer and an output layer; and the first network layer is located between the input layer and the output layer, and is connected to the output layer.

Clause 17. A model construction method, comprising: constructing at least one network layer to obtain a first neural network model for lung lobe segmentation; wherein the at least one network layer includes at least one first network layer; and the first network layer is configured to add an additional channel with coordinate information to input data input to the first network layer, and to determine output data of the first network layer based on based on the input data after the additional channel is added thereto.

Clause 18. The method according to clause 17, wherein the number of the first network layers in the at least one network layer is one; the at least one network layer includes an input layer and an output layer; and the first network layer is located between the input layer and the output layer, and the first network layer is connected to the output layer.

Clause 19. A lung lobe segmentation method, comprising: inputting an image to be processed containing a lung image to a second neural network model to obtain a lung segmentation result, wherein the lung segmentation result includes an identified lung region; inputting the image to be processed to a first neural network model to obtain a lung lobe segmentation result of the image to be processed; and modifying the lung lobe segmentation result according to the lung segmentation result.

Clause 20. The method according to clause 19, wherein the lung segmentation result further includes an identified extra-pulmonary region; wherein in the lung segmentation result, a category value corresponding to respective element in the lung region is assigned a first value, and a category value corresponding to respective element in the extra-pulmonary region is assigned a second value; wherein modifying the lung lobe segmentation result according to the lung segmentation result includes: determining a calculation result of the category value corresponding to the respective element in the lung lobe segmentation result and the category value corresponding to a corresponding element in the lung segmentation result according to predefined calculation rules, as a modified category value corresponding to the respective element in the lung lobe segmentation result; and generating a modified lung lobe segmentation result according to the modified category value corresponding to the respective element in the lung lobe segmentation result; wherein the calculation rules include: a calculation result of the first value and a third value is the third value; and a calculation result of the second value and a fourth value is the second value.

Clause 21. A segmentation method, comprising: inputting an image to be processed containing a target object image to a second neural network model to obtain a target segmentation result, wherein the target segmentation result includes an identified target object region; inputting the image to be processed to a first neural network model to obtain an identified unit region corresponding to a unit constituting the target object; and modifying the unit region according to the target segmentation result.

Clause 22. The method according to clause 21, wherein the target segmentation result further includes an identified region other than the target object; a category value corresponding to respective element in the target object region in the target segmentation result is assigned a first value, and a category value corresponding to respective element in the region other than the target object is assigned a second value; and modifying the unit region according to the target segmentation result includes: determining a calculation result of the category value corresponding to the respective element in the unit region and the category value corresponding to a corresponding element in the target segmentation result according to predefined calculation rules, as a modified category value corresponding to the respective element in the unit region; and generating a modified unit region according to the modified category value corresponding to the respective element in the unit region; wherein the calculation rules include: a calculation result of the first value and a third value is the third value; a the calculation result of the second value and a fourth value is the second value.

Clause 23. A neural network system, comprising: multiple network layers for identifying a unit region corresponding to a unit constituting a target object in an image to be processed; wherein in any two of the multiple network layers that are connected to each other, input data of a downstream network layer is output data of an upstream network layer; and the multiple network layers include a first network layer; and the first network layer is configured to add an additional channel with coordinate information to input data input to the first network layer, and to determine output data of the first network layer based on the input data to which the additional channel is added.

Clause 24. The system according to clause 23, wherein the multiple network layers include an input layer and an output layer; and the first network layer is located between the input layer and the output layer, and is connected to the output layer.

Clause 25. A segmentation method, comprising: acquiring an image to be processed containing a target object image; processing the image to be processed, wherein the processing procedure includes an operation of adding an additional channel with coordinate information; and identifying a unit region corresponding to a unit constituting the target object based on a processing result of the image to be processed.

Clause 26. The method according to clause 25, wherein processing the image to be processed includes: performing feature extraction on the image to be processed to obtain a first feature map; and adding the additional channel to the first feature map to obtain a second feature map.

Clause 27. The method according to clause 26, wherein processing the image to be processed further includes: performing feature extraction on the second feature map to obtain a third feature map.

Clause 28. The method according to any one of clauses 25 to 27, wherein the step of adding the additional channel is implemented by a first network layer in a first neural network model.

Clause 29. The method according to clause 28, wherein the first neural network model comprises an input layer and an output layer; and the first network layer is located between the input layer and the output layer, and is connected to the output layer.

Clause 30. The method according to any one of clauses 25 to 27, further comprising: inputting the image to be processed to a second neural network model to obtain a target segmentation result, wherein the target segmentation result includes the identified target object region; and modifying the unit region according to the target segmentation result.

Clause 31. The method according to clause 30, wherein the target segmentation result further includes an identified region other than the target object; and prior to the step of processing the image to be processed, the method further includes: altering a gray value of a first region corresponding to the region other than the target object in the image to be processed to enhance a difference between the first region and a second region corresponding to the target object region in the image to be processed.

Clause 32. An electronic device, comprising: a memory and a processor, wherein: the memory is configured to store a program; and the processor is coupled to the memory, and is configured to execute the program stored in the memory, so as to: determine a first neural network model; and input an image to be processed containing a lung image to the first neural network model to obtain a lung lobe segmentation result of the image to be processed; wherein a first network layer in the first neural network model is configured to add an additional channel with coordinate information to input data input to the first network layer, and to determine output data of the first network layer based on the input data after the additional channel is added thereto.

Clause 33. An electronic device, comprising: a memory and a processor, wherein: the memory is configured to store a program; and the processor is coupled to the memory, and is configured to execute the program stored in the memory, so as to: input a sample image containing a lung image to a first neural network model to obtain a lung lobe segmentation prediction result of the sample image; and perform parameter optimization on the first neural network model according to the lung lobe segmentation prediction result and a lung lobe segmentation ground truth result of the sample image; wherein the first neural network model is configured for lung lobe segmentation; the first network layer in the first neural network model is configured to add an additional channel with coordinate information to input data input to the first network layer, and to determine output data of the first network layer based on the input data to which the additional channel is added.

Clause 34. An electronic device, comprising: a memory and a processor, wherein: the memory is configured to store a program; and the processor is coupled to the memory, and is configured to execute the program stored in the memory, so as to: construct at least one network layer to obtain a first neural network model for lung lobe segmentation; wherein the at least one network layer includes at least one first network layer; and the first network layer is configured to add an additional channel with coordinate information to input data input to the first network layer, and to determine output data of the first network layer based on the input data after the additional channel is added thereto.

Clause 35. An electronic device, comprising: a memory and a processor, wherein: the memory is configured to store a program; and the processor is coupled to the memory, and is configured to execute the program stored in the memory, so as to: input an image to be processed containing a lung image to a second neural network model to obtain a lung segmentation result, wherein the lung segmentation result includes an identified lung region; input the image to be processed to a first neural network model to obtain a lung lobe segmentation result of the image to be processed; and modify the lung lobe segmentation result according to the lung segmentation result.

Clause 36. An electronic device, comprising: a memory and a processor, wherein: the memory is configured to store a program; and the processor is coupled to the memory, and is configured to execute the program stored in the memory, so as to: input an image to be processed containing a target object image to a second neural network model to obtain a target segmentation result, wherein the target segmentation result includes an identified target object region; input the image to be processed to a first neural network model to obtain an identified unit region corresponding to a unit constituting the target object; and modifying the unit region according to the target segmentation result.

Clause 37. An electronic device, comprising: a memory and a processor, wherein: the memory is configured to store a program; and the processor is coupled to the memory, and is configured to execute the program stored in the memory, so as to: acquire an image to be processed containing a target object image; process the image to be processed, wherein the processing procedure includes an operation of adding an additional channel with coordinate information; and identifying a unit region corresponding to a unit constituting the target object based on a processing result of the image to be processed.

What is claimed is:

1. A method, comprising:
determining a first neural network model; and
inputting an image to be processed containing a lung image to the first neural network model to obtain a lung lobe segmentation result of the image to be processed;
wherein the first network layer in the first neural network model is configured to add an additional channel with coordinate information to input data input to the first network layer, and to determine output data of the first network layer based on the input data after the additional channel is added thereto.

2. The method of claim 1, wherein the input data is a multi-channel feature map; and the multi-channel feature map includes a first channel map;
the method further comprising:
generating coordinate information of a respective element according to a position of the respective element in the first channel map; and
generating the additional channel according to the position of the respective element in the first channel map and the coordinate information of the respective element.

3. The method of claim 2, wherein the image to be processed is a three-dimensional image; the first channel map is a three-dimensional channel map; and the coordinate information includes a first coordinates on a first axis, a second coordinate on a second axis, and a third coordinate on a third axis; and the first axis, the second axis, and the third axis intersect one with another; and
generating the additional channel according to the position of the respective element in the first channel map and the coordinate information of the respective element includes:
generating a first additional channel corresponding to the first axis according to the position of the respective element in the first channel map and the first coordinate of the respective element;
generating a second additional channel corresponding to the second axis according to the position of the respective element in the first channel map and the second coordinate of the respective element; and
generating a third additional channel corresponding to the third axis according to the position of the respective element in the first channel map and the third coordinate of the respective element.

4. The method of claim 2, wherein generating the coordinate information of the respective element according to the position of the respective element in the first channel map includes:
performing normalization processing on position information of the respective element in the first channel map to obtain the coordinate information of the respective element.

5. The method of claim 1, further comprising:
inputting the image to be processed to a second neural network model to obtain a lung segmentation result, wherein the lung segmentation result includes an identified lung region and/or an identified extra-pulmonary region; and
modifying the lung lobe segmentation result according to the lung segmentation result.

6. The method of claim 5, wherein modifying the lung lobe segmentation result according to the lung segmentation result includes:
when the lung segmentation result includes the identified lung region, determining an extra-pulmonary misclassified region in the lung lobe segmentation result according to the lung region;
when the lung segmentation result includes the identified extra-pulmonary region, determining an extra-pulmonary misclassified region in the lung lobe segmentation result according to the identified extra-pulmonary region; and
in the lung lobe segmentation result, modifying a category of the extra-pulmonary misclassified region to an extra-pulmonary region category to obtain a modified lung lobe segmentation result.

7. The method of claim 5, wherein the lung segmentation result includes the identified lung region and the identified extra-pulmonary region; a category value corresponding to a respective element in the lung region in the lung segmentation result is assigned a first value; and a category value corresponding to a respective element in the identified extra-pulmonary region is assigned a second value; and
modifying the lung lobe segmentation result according to the lung segmentation result includes:
determining a calculation result of the category value corresponding to the respective element in the lung segmentation result and a category value corresponding to corresponding element in the lung segmentation result according to predefined calculation rules, as a modified category value corresponding to the respective element in the lung lobe segmentation result; and
generating a modified lung lobe segmentation result according to the modified category value corresponding to the respective element in the lung lobe segmentation result;
wherein the calculation rules include:
a calculation result of the first value and a third value is the third value; and
a calculation result of the second value and a fourth value is the second value.

8. The method of claim 5, wherein the lung lobe segmentation result includes the identified lung region and the identified extra-pulmonary region; and
prior to inputting the image to be processed to the first neural network model, the method further includes:
altering a gray value of a first region corresponding to the identified extra-pulmonary region in the image to be processed to enhance a difference between the first region and a second region corresponding to the lung region in the image to be processed.

9. The method of claim 8, wherein altering the gray value of the first region corresponding to the identified extra-pulmonary region in the image to be processed to enhance the difference between the first region and the second region corresponding to the lung region in the image to be processed includes:
uniformly setting the gray value of the first region in the image to be processed to a set value greater than a first preset threshold.

10. The method of claim 1, wherein the first neural network model further includes an input layer and an output layer; and
the first network layer is located between the input layer and the output layer, and is connected to the output layer.

11. The method of claim 1, wherein determining the output data of the first network layer based on the input data after the additional channel is added thereto includes:
performing feature extraction on the input data after the additional channel is added thereto to obtain a third feature map as the output data of the first network layer.

12. An apparatus, comprising:
one or more processors; and
one or more memories storing thereon computer-readable instructions that, when executed by the one or more processors, cause the one or more processors to perform acts comprising:
inputting a sample image containing a lung image to a first neural network model to obtain a lung lobe segmentation prediction result of the sample image; and
performing parameter optimization on the first neural network model according to the lung lobe segmentation prediction result and a lung lobe segmentation ground truth result of the sample image;
wherein the first neural network model is configured for lung lobe segmentation; a first network layer in the first neural network model is configured to add an additional channel with coordinate information to input data input to the first network layer, and to determine output data of the first network layer based on the input data to which the additional channel is added.

13. The apparatus of claim 12, wherein the lung lobe segmentation prediction result includes a lung lobe classification prediction result and an intrapulmonary fissure classification prediction result; and the lung lobe segmentation ground truth result includes a lung lobe classification ground truth result and an intrapulmonary fissure classification ground truth result; and
performing parameter optimization on the first neural network model according to the lung lobe segmentation prediction result and the lung lobe segmentation ground truth result of the sample image includes:
calculating a first loss value according to the lung lobe classification prediction result and the lung lobe classification ground truth result;
calculating a second loss value according to the intrapulmonary fissure classification prediction result and the intrapulmonary fissure classification ground truth result; and
performing parameter optimization on the first neural network model integrate the first loss value and the second loss value based on an integration of the first loss value and the second loss value.

14. The apparatus of claim 13, wherein calculating the first loss value according to the lung lobe classification prediction result and the lung lobe classification ground truth result includes:
executing a first Dice coefficient loss function to obtain the first loss value, with the lung lobe classification prediction result and the lung lobe classification ground truth result as inputs of the first Dice coefficient loss function; and
wherein calculating the second loss value according to the intrapulmonary fissure classification prediction result and the intrapulmonary fissure classification ground truth result includes:
executing a second Dice coefficient loss function to obtain the second loss value, with the intrapulmonary fissure classification prediction result and the intrapulmonary fissure classification ground truth result as inputs of the second Dice coefficient loss function.

15. The apparatus of claim 12, prior to inputting the sample image containing the lung image to the first neural network model, the acts further comprising:
inputting the sample image to a second neural network model to obtain a lung segmentation result, wherein the lung segmentation result includes an identified lung region and an identified extra-pulmonary region; and
altering a gray value of a first region corresponding to the identified extra-pulmonary region in the sample image to enhance a difference between the first region and a second region corresponding to the lung region in the sample image.

16. The apparatus of claim 12, wherein the first neural network model further includes an input layer and an output layer; and
the first network layer is located between the input layer and the output layer, and is connected to the output layer.

17. One or more computer-readable media, stored thereon computer-readable instructions that, when executed by one or more processors, cause the one or more processors to perform acts comprising:
inputting an image to be processed containing a target object image to a second neural network model to obtain a target segmentation result, wherein the target segmentation result includes an identified target object region;
inputting the image to be processed to a first neural network model to obtain an identified unit region corresponding to a unit constituting the target object; and
modifying the unit region according to the target segmentation result.

18. The one or more computer-readable media of claim 17, wherein the target segmentation result further includes an identified region other than the target object.

19. The one or more computer-readable media of claim 18, wherein a category value corresponding to a respective element in the target object region in the target segmentation result is assigned a first value, and a category value corresponding to a respective element in the region other than the target object is assigned a second value.

20. The one or more computer-readable media of claim 19, wherein modifying the unit region according to the target segmentation result includes:
determining a calculation result of the category value corresponding to the respective element in the unit region and the category value corresponding to a corresponding element in the target segmentation result according to predefined calculation rules, as a modified category value corresponding to the respective element in the unit region; and
generating a modified unit region according to the modified category value corresponding to the respective element in the unit region;
wherein the calculation rules are: a calculation result of the first value and a third value is the third value; and a calculation result of the second value and a fourth value is the second value.
